(19) United States
(12) Patent Application Publication
(10) Pub. No.: US 2008/0161254 A1
(43) Pub. Date: Jul. 3, 2008

(54) HCV INHIBITORS

(75) Inventors: Michael J. Green, Half Moon Bay, CA (US); Jeffrey M. Dener, Millbrae, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ViroBay, Inc., Menlo Park, CA (US)

(21) Appl. No.: 11/968,475

(22) Filed: Jan. 2, 2008

Related U.S. Application Data
(60) Provisional application No. 60/878,544, filed on Jan. 3, 2007.

(51) Int. Cl. A61K 31/7056; C07D 495/04; A61P 31/12; A61K 31/4365

(52) U.S. Cl. 514/43; 546/114; 514/301

(57) ABSTRACT

The present invention is directed to compounds that are antiviral agents. Specifically, the compounds of the present invention inhibit replication of HCV and are therefore useful in treating hepatitis C infections. The present invention is also directed to pharmaceutical compositions comprising these compounds and processes for preparing them.

### CROSS-REFERENCES TO RELATED APPLICATIONS

This application claims the benefit under 35 USC § 119(e) of provisional application Ser. No. 60/878,544, filed Jan. 3, 2007, the content of which is incorporated by reference in its entirety.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

1. Field of the Invention

The present invention is directed to compounds that inhibit HCV replication and are therefore useful in treating hepatitis C. The present invention is also directed to pharmaceutical compositions comprising these compounds and processes for preparing them.

2. State of the Art

Hepatitis C virus (HCV) is a (+)-sense single-standed RNA virus that is a major cause of non-A, non-B hepatitis worldwide. A large percentage of people infected with HCV develop chronic liver disease. This chronic hepatitis C infection, in turn, makes them at high risk for developing serious liver diseases such as liver cirrhosis, hepatocellular carcinoma and terminal liver disease leading to death. Currently, hepatitits C infections are treated with either injectable interferon or with pegylated forms of interferon such as PEG-Intron® and Pegasys®, alone or in combination with Ribavirin. These therapies, however, induce severe side effects such as retinopathy, thyroiditis, acute pancreatitis, depression. Therefore, there is a need for safe, oral drug for the treatment of hepatitis C infections. The present invention fulfils this and related needs.

### BRIEF SUMMARY OF THE INVENTION

In one aspect, this invention is directed to a compound of Formula (I):

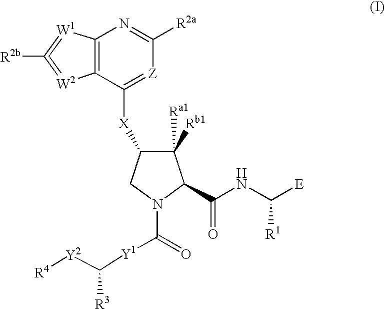

wherein

E is selected from —C(O)C(O)NR⁵R⁶, —C(O)CF₂C(O)NR⁵R⁶, —C(O)CF₂C(O)OR⁵, —C(O)C(O)R⁷, —C(O)CF₂R⁸, —C(O)R⁹, —C(O)C(O)OR¹⁰, —C(O)NR¹¹R¹², and —B(OR¹³)₂; wherein R⁵, R⁶, R⁷, R⁹, R¹⁰, R¹¹, R¹² and each R¹³ are independently selected from hydrogen, alkyl, alkenyl, alkynyl, cycloalkyl, cycloalkylalkyl, aryl, aralkyl, heteroaryl, heteroaralkyl, heterocyclyl, and heterocyclylalkyl; wherein R⁸ is selected from halo, alkyl, alkenyl, alkynyl, cycloalkyl, cycloalkylalkyl, aryl, aralkyl, heteroaryl, heteroaralkyl, heterocyclyl, and heterocyclylalkyl; wherein each aliphatic, alicyclic or aromatic moiety in R⁵, R⁶, R⁷, R⁸, R⁹, R¹⁰, R¹¹, R¹² or R¹³ is, optionally, independently substituted with one, two, or three R^{a}; wherein each R^{a} is independently selected from hydroxy, alkoxy, haloalkoxy, aryloxy, heteroaryloxy, alkylthio, arylthio, heteroarylthio, amino, monosubstituted amino, disubstituted amino, alkylsulfonyl, arylsulfonyl, carboxy, alkoxycarbonyl, alkenyloxycarbonyl, alkynyloxycarbonyl, alkenylaminocarbonyl, acylamino, aminocarbonyl, halo, and cyano; wherein each aromatic or alicyclic moiety in R^{a} is optionally, independently substituted with one, two, or three R^{b}; wherein each R^{b} is independently selected from alkyl, alkoxy, halo, haloalkyl, haloalkoxy, cyano, carboxy, and carboxyalkyl; and wherein the two groups R⁵ and R⁶ or the two groups R¹¹ and R¹², in combination with the nitrogen to which the groups are attached, optionally form a five- to seven-membered heterocyclic or heteroaromatic ring;

W¹ and W² are each independently selected from CR^{2c},O, S, N, and NR^{2d}; wherein the dashed line indicates the presence of one double bond, either between W¹ and CR^{2b} or between W² and CR^{2b};

X is selected from —O—, —NR^{2e}—, —S—, —SO—, and —SO₂—;

Z is selected from CH and N;

R¹ is independently selected from hydrogen, alkyl, haloalkyl, alkenyl, alkynyl, cycloalkyl, cycloalkylalkyl, aryl, aralkyl, heteroaryl, heteroaralkyl, heterocyclyl, and heterocyclylalkyl; wherein each aliphatic, alicyclic or aromatic moiety in R¹ is optionally, independently substituted with one or two R^{c}; wherein each R^{c} is independently selected from hydroxy, alkoxy, aryloxy, heteroaryloxy, alkylthio, arylthio, heteroarylthio, amino, monosubstituted amino, disubstituted amino, alkylsulfonyl, arylsulfonyl, carboxy, alkoxycarbonyl, acylamino, aminocarbonyl, halo, and cyano; wherein each aromatic or alicyclic moiety in R^{c} is, optionally, independently substituted with one, two, or three R^{d}; and wherein each R^{d} is independently selected from the group consisting of alkyl, alkoxy, halo, haloalkyl, haloalkoxy, cyano, carboxy, and carboxyalkyl;

R^{2a}, R^{2b}, R^{2c}, R^{2d}, and R^{2e} are each independently selected from the group consisting of hydrogen, halo, cyano, alkyl, haloalkyl, alkenyl, alkynyl, —C(O)NR¹⁴R¹⁵, —OR¹⁴, —C(O)R¹⁴, —C(O)OR¹⁴, —OC(O)R¹⁴, —NR¹⁴C(O)R¹⁵, —NR¹⁴C(O)NR¹⁵R¹⁶, —NR¹⁴R¹⁵, —NR¹⁴ OR¹⁵, —SO₂NR¹⁴R¹⁵, NR¹⁴SO₂R¹⁵, aryl, heteroaryl, heterocyclyl, and cycloalkyl, provided that one of or both R^{2a} and R^{2b} are other than hydrogen, and further provided that both R^{2d} and R^{2e} are other than a member of the group consisting of halo, —OR¹⁴, NR¹⁴OR¹⁵, —OC(O)R¹⁴, —NR¹⁴C(O)R¹⁵, —NR¹⁴C(O)NR¹⁵R¹⁶, —NR¹⁴R¹⁵, —NR¹⁴SO₂R¹⁵, and NR¹⁴SO₂R¹⁵; wherein each R¹⁴, R¹⁵, or R¹⁶ is independently selected from the group consisting of hydrogen, alkyl, cycloalkyl, cycloalkoxy, alkenyl, alkynyl, aryl, and heterocyclyl; wherein each heterocyclic, alicyclic or aromatic moiety in R¹⁴, R¹⁵, or R¹⁶ is, optionally, independently substituted with one, two, three, four, or five R^{e}; wherein each R^{e} is independently selected from the group consisting of alkyl, alkenyl, alkynyl, halo, hydroxyl, alkoxy, cycloalkyloxy, alkenyloxy, alkynyloxy, aryloxy, heteroaryloxy, cycloalkyloxy, amino, monosubstituted amino, disubstituted amino, alkylthio, arylthio, heteroarylthio, trifluoromethyl, sulfonyl, alkylsulfonyl, cycloalkylsulfonyl, arylsulfonyl, heteroarylsulfonyl, alkenylsulfonyl, alkynylsulfonyl, and heterocyclylsulfonyl; wherein each R^{e} is, optionally, independently substituted with one, two, or three R^{f}; and wherein each R^{f} is independently selected from the group consisting of alkyl, alkoxy, halo, haloalkyl, haloalkoxy, cyano, carboxy, and carboxyalkyl;

R³ is selected from the group consisting of hydrogen, alkyl, haloalkyl, alkenyl, alkynyl, cycloalkyl, cycloalkylalkyl, aryl, aralkyl, heteroaryl, heteroaralkyl, heterocyclyl, and heterocyclylalkyl; wherein each aliphatic, alicyclic or aromatic moiety in R³ is, optionally, independently substituted with one or two R^{g}; wherein each R^{g} is independently selected from the group consisting of hydroxy, alkoxy, haloalkoxy, aryloxy, heteroaryloxy, amino, monosubstituted amino, disubstituted amino, alkylthio, arylthio, heteroarylthio, alkylsulfonyl, arylsulfonyl, carboxy, alkoxycarbonyl, acylamino, aminocarbonyl, halo, and cyano; wherein each aromatic or alicyclic moeity in R^{g} is, optionally, substituted with one, two, or three R^{h}; and wherein each R^{h} is independently selected from the group consisting of alkyl, alkoxy, halo, haloalkyl, haloalkoxy, carboxy, or carboxyalkyl;

Y¹ is NR¹⁷ or a bond; wherein R¹⁷ is selected from the group consisting of hydrogen, alkyl, haloalkyl, cycloalkyl, and halocycloalkyl;

Y² is —C(O)NH—, —OC(O)NH—, —NR¹⁸C(O)NH—, or —NR¹⁸C(O)O—; wherein each R¹⁸ is selected from the group consisting of hydrogen, alkyl, aryl, heteroaryl, or heterocyclyl; wherein each alkyl moiety in R¹⁸ is, optionally, independently substituted with one, two, or three Rⁱ groups; wherein each Rⁱ is independently selected from the group consisting of halo, hydroxyl, alkoxy, amino, monosubstituted amino, disubstituted amino, aryl, heteroaryl, and heterocyclyl; wherein each aromatic, heteroaromatic, or heterocyclic moeity in R¹⁸ or Rⁱ is, optionally, independently substituted with one, two, or three R^{j}; and wherein each R^{j} is chosen from the group consisting of halo and alkyl;

R⁴ is:
(i) alkyl, provided that Y¹ is a bond; Y² is —OC(O)NH—, —NR¹⁸—C(O)NH—, or —NR¹⁸C(O)O— and one, two, or three R¹ are other than hydrogen;
(ii) selected from the group consisting of cycloalkyl, cycloalkylalkyl, aryl, aralkyl, heteroaryl, heteroaralkyl, heterocyclyl, and heterocyclylalkyl, provided that one or two members from the group consisting of R^{2a} and R^{2b} are independently selected heteroaryl when Y¹ is a bond; or
(iii) alkyl, provided that Y¹ is a bond; Y² is —C(O)NH— or —SO₂NH—, and one or two members from the group consisting of R^{2a} and R^{2b} are independently selected heteroaryl;
   wherein each of the aromatic or alicyclic moieties in R⁴ is, optionally, independently substituted with one, two, or three R^{k}; wherein each R^{k} is independently selected from the group consisting of alkyl, alkoxy, halo, haloalkyl, haloalkoxy, cyano, carboxy, carboxyalkyl, hydroxyalkyl, alkoxyalkyl, aminoalkyl, alkylsulfonyl, alkylcarbonyl, aryl, aralkyl, arylsulfonyl, arylcarbonyl, aryloxycarbonyl, aminosulfonyl, aminocarbonyl, heteroaryl, heteroaralkyl, heteroarylsulfonyl, heteroarylcarbonyl, heteroaryloxycarbonyl, heterocyclyl, heterocyclylalkyl, heterocyclylsulfonyl, heterocyclylcarbonyl, heterocyclyloxycarbonyl, monosubstituted amino, and disubstituted amino; wherein each aromatic or alicyclic ring in R^{k} is, optionally, independently substituted with one, two, or three R^{m}; wherein each R^{m} is independently selected from the group consisting of alkyl, alkoxy, halo, haloalkyl, haloalkoxy, hydroxy, carboxy, alkoxycarbonyl, monosubstituted amino, disubstituted amino, and acylamino;

R^{a1} and R^{b1} are each independently selected from the group consisting of hydrogen, alkyl, cycloalkyl, and alkenyl; wherein each of the alkyl and cycloalkyl moieties in R^{a1} and R^{b1} are, optionally, independently substituted with one, two, or three Rⁿ moieties; wherein each Rⁿ moiety is independently selected from the group consisting of alkoxy, alkylthio, alkylsulfonyl, cycloalkyl, and halo;

wherein the compound has a molecular weight greater than 400 atomic mass units and less than 1100 atomic mass units; or

a pharmaceutically acceptable salt or solvate thereof.

For the sake of clarity, it is pointed out that the point of attachment of the Y groups to the R⁴ group as follows: R⁴C(O)NH—, R⁴OC(O)NH—, R⁴NR¹⁴—C(O)NH—, or R⁴NR¹⁴C(O)O—.

In a second aspect, this invention is directed to a pharmaceutical composition comprising a compound of Formula (I) or a pharmaceutically acceptable salt thereof in admixture with one or more pharmaceutically acceptable excipient.

In a third aspect, this invention is directed to a method for treating hepatitis C in an animal which method comprises administering to the animal a pharmaceutical composition comprising a therapeutically effective amount of a compound of Formula (I) or a pharmaceutically acceptable salt thereof in admixture with one or more pharmaceutically acceptable excipient.

In a fourth aspect, this invention is directed to processes for preparing compounds of Formula (I).

### BRIEF DESCRIPTION OF THE DRAWINGS

FIG. 1 provides synthesis Schemes 1 and 2, as described in detail below.

FIG. 2 provides synthesis Schemes 3 and 4, as described in detail below.

FIG. 3 provides synthesis Schemes 5, 6 and 7, as described in detail below.

FIG. 4 provides synthesis schemes for Reaction intermediates A and B, as described in detail below.

FIG. 5 provides Example 1 Scheme, as described in detail below.

FIG. 6 provides Example 2 Scheme and Example 3 Scheme, as described in detail below.

FIG. 7 provides Example 4 Scheme, as described in detail below.

### DETAILED DESCRIPTION OF THE INVENTION

### Definitions

Unless otherwise stated, the following terms used in the specification and claims are defined for the purposes of this Application and have the following meanings.

“Alicyclic” means a moiety characterized by arrangement of the carbon atoms in closed non-aromatic ring structures e.g., cycloalkyl and heterocyclyl rings as defined herein.

“Aliphatic” means alkyl, alkenyl, or alkynyl radicals as defined herein

“Alkyl” represented by itself means a straight or branched, saturated aliphatic radical containing one to eight carbon atoms, unless otherwise indicated e.g., alkyl includes methyl, ethyl, propyl, isopropyl, butyl, sec-butyl, isobutyl, tert-butyl, and the like.

“Alkylcarbonylamino” refers to a —NHC(O)R radical where R is an alkyl group as defined above e.g., methylcarbonylamino, ethylcarbonylamino, and the like.

“Alkylene”, unless indicated otherwise, means a straight or branched, saturated aliphatic, divalent radical having the number of one to six carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Alkenyl” means a linear monovalent hydrocarbon radical of two to six carbon atoms or a branched monovalent hydrocarbon radical of three to six carbon atoms containing one or two double bonds e.g., ethenyl, propenyl (including all isomeric forms), 1-methylpropenyl, butenyl (including all isomeric forms), or pentenyl (including all isomeric forms), and the like.

“Alkenyloxycarbonyl” refers to a —C(O)OR radical where R is an alkenyl group as defined above e.g., 3-propen-1-yloxycarbonyl, and the like.

“Alkenylaminocarbonyl” refers to a —C(O)NHR radical where R is an alkenyl group as defined above e.g., 3-propen-1-ylaminocarbonyl, and the like.

“Alkynyl” means a linear monovalent hydrocarbon radical of two to six carbon atoms or a branched monovalent hydrocarbon radical of three to six carbon atoms containing one or two triple bonds e.g., ethynyl, propynyl (including all isomeric forms), 1-methylpropynyl, butynyl (including all isomeric forms), or pentynyl (including all isomeric forms), and the like.

“Alkynyloxycarbonyl” refers to a —C(O)OR radical where R is an alkynyl group as defined above e.g., 3-propyn-1-yloxycarbonyl, and the like.

“Alkylthio” means an —SR radical where R is alkyl as defined herein, e.g., methylthio, ethylthio, propylthio, or butylthio, and the like.

“Alkylsulfonyl” means —SO₂R radical where R is alkyl as defined herein e.g., methylsulfonyl, ethylsulfonyl, and the like.

“Alkoxy” refers to a —OR radical where R is an alkyl group as defined above e.g., methoxy, ethoxy, and the like.

“Alkoxycarbonylamino” refers to a —NHC(O)OR radical where R is an alkyl group as defined above e.g., methoxycarbonylamino, ethoxycarbonylamino, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxy-ethyl, 1-, 2-, or 3-methoxypropyl, 2-ethoxyethyl, and the like.

“Alkoxycarbonyl” refers to a —C(O)OR radical where R is an alkyl group as defined above e.g., methoxycarbonyl, ethoxycarbonyl, and the like.

“Amino” means a —NH₂ radical.

“Alkylamino” means a radical —NHR where R is alkyl as defined herein, e.g., methylamino, ethylamino, n-, iso-propylamino, n-, iso-, tert-butylamino, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, acyl, hydroxyalkyl, alkoxyalkyl, aryl, aralkyl, heteroaryl, heteroaralkyl or heterocyclylalkyl and R′ is hydrogen, alkyl, hydroxyalkyl, alkoxyalkyl, aryl, aralkyl, heteroaryl, heteroaralkyl, heterocyclylalkyl, cycloalkyl, cycloalkylalkyl, aminocarbonyl, or aminosulfonyl as defined herein e.g., aminomethyl, methylaminoethyl, dimethylaminoethyl, 1,3-diaminopropyl, acetylaminopropyl, and the like.

“Acyl” refers to a —COR radical where R is hydrogen, alkyl, haloalkyl, cycloalkyl, aryl, aralkyl, heteroaryl, heteroaralkyl, or heterocyclyl as defined herein, e.g., formyl, acetyl, trifluoroacetyl, benzoyl, piperazin-1-ylcarbonyl, and the like. When R is alkyl it is referred to in this application as alkylcarbonyl. When R is aryl it is referred to in this application as arylcarbonyl. When R is heteroaryl it is referred to in this application as heteroarylcarbonyl. When R is heterocyclyl it is referred to in this application as heterocyclylcarbonyl.

“Acylamino” refers to a —NRCOR′ radical where R is hydrogen or alkyl and R′ is hydrogen, alkyl, haloalkyl, cycloalkyl, aryl, aralkyl, heteroaryl, heteroaralkyl, or heterocyclyl as defined herein, e.g., formyl, acetyl, trifluoroacetyl, benzoyl, piperazin-1-ylcarbonyl, and the like.

“Aminocarbonyl” means —CONRR′ radical where R and R′ are independently selected from hydrogen, alkyl, aryl, aralkyl, heteroaryl, heteroaralkyl, or heterocyclylalkyl or R and R′ together with the nitrogen atom to which they are attached form heterocycloamino as defined herein.

“Aminosulfonyl” means —SO₂NRR′ radical where R and R′ are independently selected from hydrogen, alkyl, aryl, aralkyl, heteroaryl, heteroaralkyl, or heterocyclylalkyl or R and R′ together with the nitrogen atom to which they are attached form heterocycloamino as defined herein.

“Animal” includes humans, non-human mammals (e.g., dogs, cats, rabbits, cattle, horses, sheep, goats, swine, deer, and the like) and non-mammals (e.g., birds, and the like).

“Aromatic” refers to a moiety wherein the constituent atoms make up an unsaturated ring system, all atoms in the ring system are sp² hybridized and the total number of pi electrons is equal to 4n+2.

“Aryl” refers to a monocyclic or fused bicyclic ring assembly containing 6 to 10 ring carbon atoms wherein each ring is aromatic e.g., phenyl or naphthyl.

“Aryloxy” refers to a —O—R radical where R is aryl as defined above e.g., phenoxy, napthyloxy, and the like.

“Aryloxycarbonyl” refers to a —C(O)OR radical where R is aryl as defined above e.g., phenyloxycarbonyl, naphthyloxycarbonyl, and the like.

“Aralkyl” refers to a -(alkylene)-R radical where R is aryl as defined above e.g., benzyl, phenethyl, and the like.

“Arylthio” means an —SR radical where R is aryl as defined herein, e.g., phenylthio or naphthylthio.

“Arylsulfonyl” means an —SO₂R radical where R is aryl as defined herein, e.g., phenylsulfonyl or naphthylsulfonyl.

“Carboxy” refers to —C(O)OH radical.

“Carboxyalkyl” means an alkyl radical, as defined herein, substituted with at least one, preferably one or two, —C(O)OH group(s), e.g., carboxymethyl, carboxyethyl, 1-, 2-, or 3-carboxypropyl, and the like.

“Cycloalkyl” refers to a monovalent saturated monocyclic ring containing three to eight ring carbon atoms e.g., cyclopropyl, cyclobutyl, cyclopentyl, cyclohexyl, and the like.

“Cycloalkylalkyl” refers to a -(alkylene)-R radical where R is cycloalkyl as defined above e.g., cyclopropylmethyl, cyclobutylethyl, cyclobutylmethyl, and the like.

“Cycloalkyloxy” refers to a —OR radical where R is cycloalkyl as defined above e.g., cyclopropyloxy, cyclopentyloxy, cyclohexyloxy, and the like.

“Cycloalkyloxycarbonylamino” refers to a —NHC(O)OR radical where R is cycloalkyl as defined above e.g., cyclopropyloxycarbonylamino, cyclopentyloxycarbonylamino, and the like.

“Cycloalkylalkyloxycarbonylamino” refers to a —NHC(O)OR radical where R is cycloalkylalkyl as defined above e.g., cyclopropylmethyloxycarbonylamino, cyclopentylmethyloxycarbonylamino, and the like.

“Disease” specifically includes any unhealthy condition of an animal or part thereof and includes an unhealthy condition that may be caused by, or incident to, medical or veterinary therapy applied to that animal, i.e., the “side effects” of such therapy.

“Dialkylamino” means a radical —NRR′ where R and R′ are independently alkyl as defined herein, e.g., dimethylamino, diethylamino, N,N-methylpropylamino or N,N-methylethylamino, and the like.

“Disubstituted amino” means a radical —NRR′ where R and R′ are independently selected from alkyl, cycloalkyl, cycloalkylalkyl, hydroxyalkyl, alkoxyalkyl, aminoalkyl, aryl, aralkyl, heteroaryl, or heteroaralkyl as defined herein, e.g., dimethylamino, diethylamino, N,N-methylpropylamino or N,N-methylethylamino, methylphenylamino, and the like. Dialkylamino is a subgroup of disubstituted amino.

“Fused heterocyclyl” means heterocyclyl radical as defined herein that is fused to an aryl or heteroaryl ring as defined herein e.g., 2,3-dihydroisoindol-1-yl, 1,2,3,4-tetrahydroisoquinolin-1-yl, and the like.

“Halo” refers to fluoro, chloro, bromo or iodo.

“Haloalkyl” refers to alkyl as defined above substituted by one or more, preferably one to seven, “halo” atoms, as such terms are defined in this Application. Haloalkyl includes monohaloalkyl, dihaloalkyl, trihaloalkyl, perhaloalkyl, and the like, e.g., chloromethyl, dichloromethyl, difluoromethyl, trifluoromethyl, 2,2,2-trifluoroethyl, perfluoroethyl, 2,2,2-trifluoro-1,1-dichloroethyl, and the like.

“Haloalkoxy” refers to a —OR radical where R is haloalkyl group as defined above e.g., trifluoromethoxy, 2,2,2-trifluoroethoxy, difluoromethoxy, and the like.

“Heteroaryl” as a group or part of a group denotes an aromatic monocyclic or bicyclic moiety of 5 to 10 ring atoms in which one or more, preferably one, two, or three, of the ring atom(s) is(are) selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, benzimidazolyl, quinolinyl, isoquinolinyl, quinazolinyl, quinoxalinyl, pyrazolyl, and the like.

“Heteroaryloxy” refers to a —O—R radical where R is heteroaryl as defined above e.g., furanyloxy, pyridinyloxy, indolyloxy, and the like.

“Heteroaryloxycarbonyl” refers to a —C(O)O—R radical where R is heteroaryl as defined above e.g., pyridinyloxycarbonyl, pyrimidinyloxycarbonyl, and the like.

“Heteroaralkyl” refers to a -(alkylene)-R radical where R is heteroaryl as defined above e.g., pyridinylmethyl, 1- or 2-furanylethyl, imidazolylmethyl, and the like.

“Heteroaralkyloxycarbonyl” refers to a —C(O)O—R radical where R is heteroaralkyl as defined above e.g., pyridinylmethyloxycarbonyl, pyrimidinylmethyloxycarbonyl, and the like.

“Heteroarylthio” means an —SR radical where R is heteroaryl as defined herein, e.g., pyridinylthio, furanylthio, thienylthio, and the like.

“Heteroarysulfonyl” means an —SO₂R radical where R is heteroaryl as defined herein, e.g., pyridinylsulfonyl, thienylsulfonyl, and the like.

“Heterocyclyl” refers to a saturated or partially unsaturated, mono or bicyclic radical of 4, 5, 6, or 7 carbon ring atoms wherein one or more, preferably one, two, or three of the ring carbon atoms are replaced by a heteroatom selected from —N═, —N—, —O—, —S—, —SO—, or —S(O)₂— and further wherein one or two ring carbon atoms are optionally replaced by a keto (—CO—) group. The heterocyclyl ring is optionally fused to cycloalkyl, aryl or heteroaryl ring as defined herein. Representative examples include, but are not limited to, imidazolidinyl, morpholinyl, thiomorpholinyl, thiomorpholino-1-oxide, thiomorpholino-1,1-dioxide, tetrahydrofuranyl, tetrahydropyranyl, tetrahydrothiopyranyl, 1-oxo-tetrahydrothiopyranyl, 1,1-dioxotetrathio-pyranyl, indolinyl, piperazinyl, piperidyl, pyrrolidinyl, pyrrolinyl, quinuclidinyl,3,4-dihydroisoquinolinyl, dihydroindolyl, and the like. When the heterocyclyl group contains at least one nitrogen ring atom it is referred to herein as “heterocycloamino” and is a subset of the heterocyclyl group as defined above.

“Heterocyclylalkyl” refers to a -(alkylene)-R radical where R is heterocyclyl as defined above e.g., pyrrolidinylmethyl, tetrahydrofuranylethyl, pyridinylmethylpiperidinylmethyl, and the like.

“Heterocyclyloxycarbonyl” refers to a —C(O)OR radical where R is heterocyclyl as defined above e.g., piperidinyloxycarbonyl, tetrahydrofuranoxycarbonyl, and the like.

“Heterocyclylsulfonyl” means an —SO₂R radical where R is heterocyclyl as defined herein, e.g., piperidin-1-ylsulfonyl, pyrrolidin-1-ylsulfonyl, and the like.

“Hydroxy” means —OH radical.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Isomers” mean compounds of Formula (I) having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center that has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (I) are meant to encompass all possible stereoisomers.

“Monosubstituted amino” means a radical —NHR where R is selected from alkyl, cycloalkyl, cycloalkylalkyl, hydroxyalkyl, alkoxyalkyl, aminoalkyl, aryl, aralkyl, heteroaryl, or heteroaralkyl as defined herein, e.g., methylamino, ethylamino, propylamino, phenylamino, benzylamino, and the like.

“Optional” or “optionally” or “may be” means that the subsequently described event or circumstance may or may not occur, and that the description includes instances where the event or circumstance occurs and instances in which it does not. For example, the phrase “wherein the aromatic ring in R^{a} is optionally substituted with one or two substituents independently selected from the group consisting of alkyl” means that the aromatic ring may or may not be substituted with alkyl in order to fall within the scope of the invention.

The present invention also includes N-oxide derivatives of a compound of Formula (I). N-oxide derivative mean a compound of Formula (I) in which a nitrogen atom is in an oxidized state (i.e., N→O) e.g., pyridine N-oxide, and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula (I) which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxy-ethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of Formula (I). Prodrug means a compound that is convertible in vivo by metabolic means (e.g. by hydrolysis) to a compound of Formula (I). For example, an ester of a compound of Formula (I) containing a hydroxy group may be convertible by hydrolysis in vivo to the parent molecule. Alternatively an ester of a compound of Formula (I) containing a carboxy group may be convertible by hydrolysis in vivo to the parent molecule. Suitable esters of compounds of Formula (I) containing a hydroxy group, are for example acetates, citrates, lactates, tartrates, malonates, oxalates, salicylates, propionates, succinates, fumarates, maleates, methylene-bis-βb-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of Formula (I) containing a carboxy group, are for example those described by Leinweber, F. J. Drug Metab. Res., 1987, 18, page 379. An especially useful class of esters of compounds of Formula (I) containing a hydroxy group, may be formed from acid moieties selected from those described by Bundgaard et al., J. Med. Chem., 1989, 32, pp 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates. It is understood that the names and illustration used in this Application to describe compounds of Formula (I) are meant to be encompassed all possible prodrugs thereof.

“Protected derivatives” means derivatives of compounds of Formula (I) in which a reactive site or sites are blocked with protecting groups. Protected derivatives of compounds of Formula (I) are useful in the preparation of compounds of Formula (I) or in themselves may be active HCV inhibitors. A comprehensive list of suitable protecting groups can be found in T. W. Greene, Protective Groups in Organic Synthesis, 3rd edition, John Wiley & Sons, Inc. 1999. It is understood that the names and illustration used in this Application to describe compounds of Formula (I) are meant to be encompassed all possible protected derivatives thereof.

“Therapeutically effective amount” means that amount which, when administered to an animal for treating a disease, is sufficient to effect such treatment for the disease.

“Treatment” or “treating” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

“Ureido” means a radical —NHCONRR′ where R is hydrogen or alkyl and R′ is hydrogen, alkyl, cycloalkyl, or cycloalkylalkyl.

The compounds of Formula I, and the intermediates and starting materials used in their preparation are named in accordance with IUPAC rules for nomenclature in which the characteristic groups have decreasing priority for citation as the principle group as follows: acids, esters, amides, etc. Alternatively, the compounds are named by ChemDraw Ultra, Version 10.0.

### PREFERRED EMBODIMENTS

Certain compounds of Formula (I) within the broadest scope set forth in the Summary of the Invention are preferred. For example:

A. A preferred group of compounds of Formula (I) is that wherein: E is —COCONHR⁶; wherein R⁶ is selected from the group consisting of hydrogen, alkyl, cycloalkyl, aralkyl, and heteroaralkyl; and wherein each aromatic ring of R⁶ is optionally substituted with one or two halo. Preferably, R⁶ is selected from the group consisting of cyclopropyl and —CH(CH₃)R; wherein R is selected from the group consisting of phenyl, 4-chlorophenyl, 2,4-dichlorophenyl, 2,4-difluorophenyl, 3,4-dichlorophenyl, 3,4-difluorophenyl, and pyridin-4-yl. More preferably, R⁶ is cyclopropyl.

B. Another preferred group of compounds of Formula (I) is that wherein: E is —COCOOR¹⁰; wherein R¹⁰ is as defined in the Summary of the Invention. Preferably, R¹⁰ is selected from the group consisting of —CH₂C≡CH, —CH₂CH═CH₂, n-propyl, 2,2-dimethylpropyl, carboxymethyl, methoxycarbonylmethyl, tert-butoxycarbonylmethyl, —CH₂C(O)OCH₂C≡CH, —CH₂C(O)OCH₂CH═CH₂, —CH₂C(O)O(CH₂)₂CH₃, —CH₂C(O)NH₂, —CH₂C(O)NHCH₃, —CH₂C(O)N(CH₃)₂, —CH₂C(O)NHCH₂CH═CH₂, and 2-phenethyl.

(a) Within the above preferred groups A and B and more preferred groups contained therein, a more preferred group of compounds is that wherein:

X is —O—;

R¹ is alkyl, optionally, independently substituted with one, two, or three moieties selected from the group consisting of alkoxy, alkylthio, alkylsulfonyl, alkenyl, alkynyl, cycloalkyl, and cycloalkylalkyl; and preferably, from the group consisting of methyl, ethyl, n-propyl, n-butyl, n-pentyl, prop-2-enyl, propyn-2-yl, 1-methylethyl, 1-methylpropyl, 2-methylpropyl, 3-methylbutyl, cyclopropyl, methoxymethyl, 2-methoxyethyl, methylthiomethyl, methylsulfonylmethyl, and cyclobutylmethyl. More preferably, R¹ is selected from the group consisting of cyclobutylmethyl, ethyl, n-propyl, and n-butyl; and

R³ is selected from the group consisting of alkyl, cycloalkyl, or aryl; and preferably, from the group consisting of 1-methylethyl, 1-methylpropyl, tert-butyl, cyclopropyl, phenyl, and cyclohexyl. More preferably, R³ is selected from the group consisting of tert-butyl and cyclohexyl.

(1) Within the groups (A), (B), A(a), and B(a) and more preferred groups contained therein, a more preferred group of compounds is that wherein:

### R^{a1} and R^{b1} are H; Y¹ is a bond; Y² is —OC(O)NH—; X is —O—; and

### R⁴ is alkyl, preferably tert-butylmethyl.

(i) Preferably, the ring system bearing R^{2a} and R^{2b} is a group of formula (a):

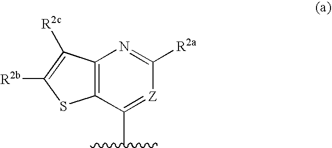

where the wavy line indicates the point of attachment to X; Z is CH or N;
R^{2a} is selected from the group consisting of aryl, heteroaryl, cycloalkyl, and heterocyclyl; wherein R^{2a} is, optionally, independently substituted with one, two, or three moieties selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkoxycarbonylamino, amino, alkylamino, dialkylamino, cycloalkylamino, cycloalkylalkylamino, and —NHCONRR′; wherein R is selected from the group consisting of hydrogen and alkyl; wherein R′ is selected from the group consisting of hydrogen, alkyl, cycloalkyl, and cycloalkylalkyl; wherein each cycloalkyl or cycloalkylalkyl moiety in R is, optionally, independently substituted with one, two or three alkyl; and

### R^{2b} and R^{2c} are independently selected from the group consisting of hydrogen, alkyl, haloalkyl, aryl, heteroaryl, halo, hydroxy, alkoxy, amino, alkylamino, dialkylamino, haloalkoxy, alkylthio and alkylsulfonyl.

Preferably, R^{2a} is a group of formula:

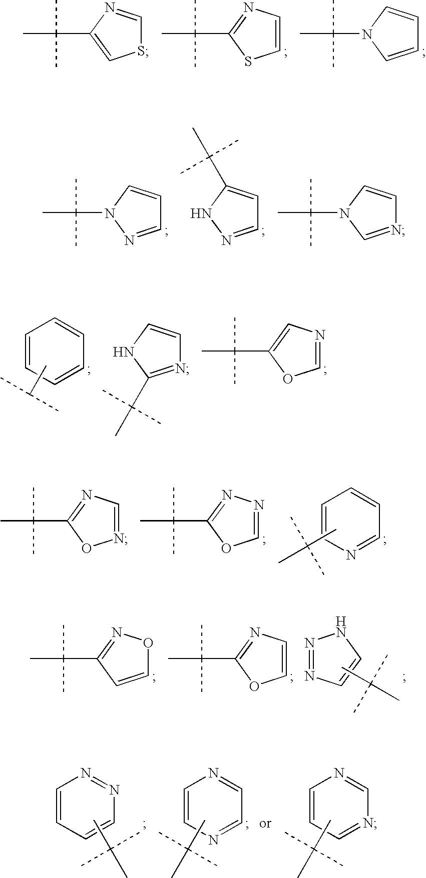

wherein the heteroaryl moiety is, optionally, substituted with one, two, or three moieties independently selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkyloxycarbonylamino, amino, alkylamino, dialkylamino, cycloalkylamino, cycloalkylalkylamino, and —NHCONRR′; wherein R is selected from the group consisting of hydrogen and alkyl; wherein R′ is selected from the group consisting of hydrogen, alkyl, cycloalkyl, and cycloalkylalkyl; and wherein each cycloalkyl or cycloalkylalkyl moiety in R′ is, optionally, independently substituted with one, two or three alkyl.

More preferably, R^{2a} is a group of formula:

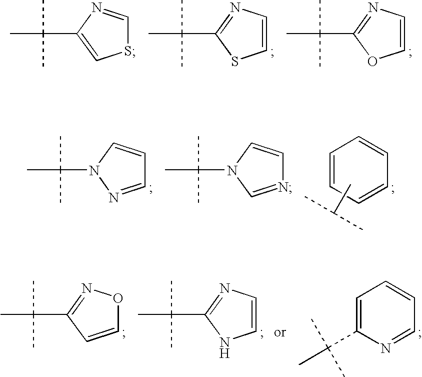

wherein the heteroaryl moiety is, optionally, substituted with one, two, or three moieties independently selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkyloxycarbonylamino, amino, alkylamino, dialkylamino, cycloalkylamino, cycloalkylalkylamino, and —NHCONRR′; wherein R is selected from the group consisting of hydrogen and alkyl; wherein R¹ is selected from the group consisting of hydrogen, alkyl, cycloalkyl, and cycloalkylalkyl; and wherein each cycloalkyl or cycloalkylalkyl moiety in R′ is, optionally, independently substituted with one, two or three alkyl.

Even more preferably, R^{2a} is:

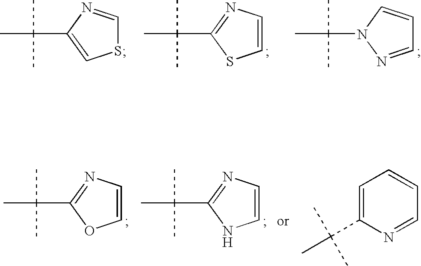

wherein the heteroaryl moiety is, optionally, substituted with one, two, or three moieties independently selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkyloxycarbonylamino, amino, alkylamino, dialkylamino, cycloalkylamino, cycloalkylalkylamino, and —NHCONRR′; wherein R′ is selected from the group consisting of hydrogen and alkyl; wherein R¹ is selected from the group consisting of hydrogen, alkyl, cycloalkyl, and cycloalkylalkyl; and wherein each cycloalkyl or cycloalkylalkyl moiety in R is, optionally, independently substituted with one, two or three alkyl.

In still other preferred embodiments of subgroup (i), R^{2a} is cycloalkyl, more preferably cyclopropyl, cyclobutyl or cyclopentyl, still more preferably, cyclopropyl. The remaining groups, R^{2b} and R^{2c}, have the meanings provided for formula (a).

Within the above preferred groups, one preferred group of compounds is that wherein:

R^{2b} and R^{2c} are each independently selected from the group consisting of hydrogen, hydroxy, methoxy, ethoxy, n-propoxy, iso-propoxy, methylamino, ethylamino, n-propylamino, isopropylamino, dimethylamino, methylethylamino, methyl(n-propyl)amino and methyl(isopropyl)amino; more preferably, from the group consisting of hydrogen, hydroxy, methoxy, and dimethylamino; and even more preferably, methoxy. Alternatively, even more preferably, hydrogen.
Another preferred group of compounds is the group wherein:
R^{2b} and R^{2c} are each independently selected from the group consisting of hydrogen, fluoro, chloro, methyl, ethynyl, methoxy, ethoxy, methylthio, and methylsulfonyl. More preferably, R^{2b} is selected from the group consisting of hydrogen, ethynyl, fluoro, chloro, methyl, methoxy, methylthio, and methylsulfonyl; and R^{2c} is hydrogen.

Most preferably, R^{2b} and R^{2c} are each independently selected from the group consisting of hydrogen and methyl.

(ii) Preferably, the ring system bearing R^{2a} and R^{2b} is a group of formula (b):

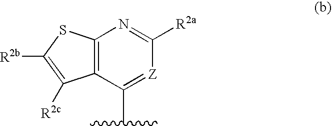

where the wavy line indicates the point of attachment to X; Z is CH or N;
R^{2a} is selected from the group consisting of aryl, heteroaryl, cycloalkyl, and heterocyclyl; wherein R^{2a} is, optionally, independently substituted with one, two, or three moieties selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkoxycarbonylamino, amino, alkylamino, dialkylamino, cycloalkylamino, cycloalkylalkylamino, and —NHCONRR′; wherein R is selected from the group consisting of hydrogen and alkyl; wherein R′ is selected from the group consisting of hydrogen, alkyl, cycloalkyl, and cycloalkylalkyl; wherein each cycloalkyl or cycloalkylalkyl moiety in R′ is, optionally, independently substituted with one, two or three alkyl; and

R^{2b} and R^{2c} are independently selected from the group consisting of hydrogen, alkyl, haloalkyl, aryl, heteroaryl, halo, hydroxy, alkoxy, amino, alkylamino, dialkylamino, haloalkoxy, alkylthio and alkylsulfonyl.

Preferably, R^{2a} is a group of formula:

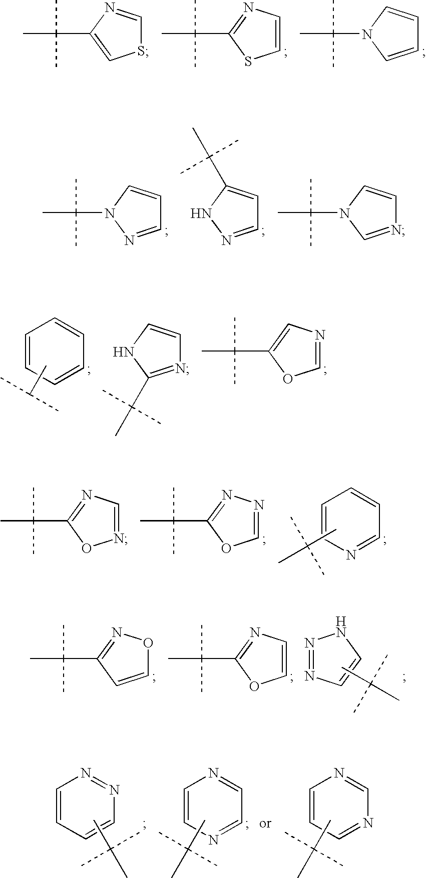

wherein the heteroaryl moiety is, optionally, substituted with one, two, or three moieties independently selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkyloxycarbonylamino, amino, alkylamino, dialkylamino, cycloalkylamino, cycloalkylalkylamino, and —NHCONRR′; wherein R is selected from the group consisting of hydrogen and alkyl; wherein R¹ is selected from the group consisting of hydrogen, alkyl, cycloalkyl, and cycloalkylalkyl; and wherein each cycloalkyl or cycloalkylalkyl moiety in R′ is, optionally, independently substituted with one, two or three alkyl.

More preferably, R^{2a} is a group of formula:

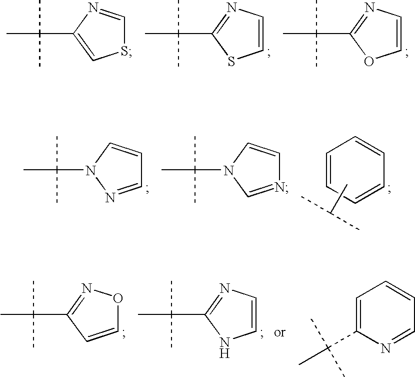

wherein the heteroaryl moiety is, optionally, substituted with one, two, or three moieties independently selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkyloxycarbonylamino, amino, alkylamino, dialkylamino, cycloalkylamino, cycloalkylalkylamino, and —NHCONRR′; wherein R is selected from the group consisting of hydrogen and alkyl; wherein R′ is selected from the group consisting of hydrogen, alkyl, cycloalkyl, and cycloalkylalkyl; and wherein each cycloalkyl or cycloalkylalkyl moiety in R′ is, optionally, independently substituted with one, two or three alkyl.

Even more preferably, R^{2a} is:

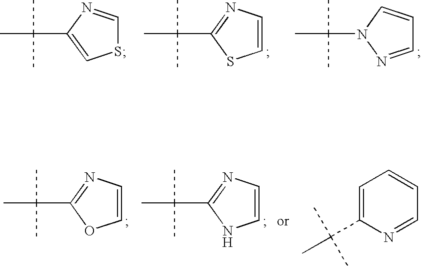

wherein the heteroaryl moiety is, optionally, substituted with one, two, or three moieties independently selected from the group consisting of halo, alkyl, cycloalkyl, cycloalkylalkyl, alkoxy, cycloalkoxy, nitro, alkylcarbonylamino, cycloalkylcarbonylamino, cycloalkylalkylcarbonylamino, alkyloxycarbonylamino, amino, alkylamino, dialkylamino, cycloalkylamino, cycloalkylalkylamino, and —NHCONRR′; wherein R is selected from the group consisting of hydrogen and alkyl; wherein R¹ is selected from the group consisting of hydrogen, alkyl, cycloalkyl, and cycloalkylalkyl; and wherein each cycloalkyl or cycloalkylalkyl moiety in R is, optionally, independently substituted with one, two or three alkyl.

In still other preferred embodiments of subgroup (i), R^{2a} is cycloalkyl; more preferably, selected from the group consisting of cyclopropyl, cyclobutyl and cyclopentyl; and still more preferably, cyclopropyl. The remaining groups, R^{2b} and R^{2c}, have the meanings provided for formula (a).

Within the above preferred groups, one preferred group of compounds is that wherein:

R^{2b} and R^{2c} are each independently selected from the group consisting of hydrogen, hydroxy, methoxy, ethoxy, n-propoxy, iso-propoxy, methylamino, ethylamino, n-propylamino, isopropylamino, dimethylamino, methylethylamino, methyl(n-propyl)amino, and methyl(isopropyl)amino; more preferably, from the group consisting of hydrogen, hydroxy, methoxy, and dimethylamino; and even more preferably methoxy. Alternatively, even more preferably hydrogen.
Another preferred group of compounds is the group wherein:
R^{2b} and R^{2c} are independently, hydrogen, fluoro, chloro, methyl, ethynyl, methoxy, ethoxy, methylthio or methylsulfonyl. More preferably, R^{2b} is hydrogen, ethynyl, fluoro, chloro, methyl, methoxy, methylthio or methylsulfonyl and R^{2c} is hydrogen.

Most preferably, R^{2b} and R^{2c} are each independently selected from the group consisting of hydrogen and methyl.

Within the above groups (i)-(ii) and more preferred groups contained therein, a more preferred group of compounds is that wherein the R^{2a} rings are optionally substituted with methyl, ethyl, n-propyl, iso-propyl, n-butyl, 1-methylpropyl, 2-methylpropyl, tert-butyl, 2,2-dimethylpropyl, 1,2-dimethylpropyl, 1,2,2-trimethylpropyl, cyclopropyl, cyclobutyl, cyclopentyl, cyclohexyl, cyclopropylmethyl, cyclobutylmethyl, cyclopentylmethyl, and cyclohexylmethyl, each of said cycloalkyl and cycloalkylalkyl rings being optionally substituted with one to three substituents independently selected from methyl or ethyl, preferably methyl.

Within the above groups (i)-(ii) and more preferred groups contained therein, a more preferred group of compounds is that wherein the R^{2a} rings are optionally substituted with amino, methylamino, ethylamino, propylamino, 1-methylethylamino, 1,1-dimethylethylamino, 2-methylpropylamino, 1-methylpropylamino, 2,2-dimethylpropylamino, 1,2-dimethylpropylamino, 1,1-dimethylpropylamino, cyclopropylamino, cyclobutylamino, cyclopentylamino, cyclohexylamino, cyclopropylmethylamino, cyclobutylmethylamino, cyclopentylmethylamino, cyclohexylmethylamino, methylcarbonylamino, ethylcarbonylamino, propylcarbonylamino, 1-methylethylcarbonylamino, 1,1-dimethylethylcarbonylamino, 2-methylpropylcarbonylamino, 1-methylpropylcarbonylamino, 2,2-dimethylpropylcarbonylamino, 1,2-dimethylpropylcarbonylamino, 1,1-dimethylpropylcarbonylamino, cyclopropylcarbonylamino, cyclobutylcarbonylamino, cyclopentylcarbonylamino, cyclohexylcarbonylamino, cyclopropylmethylcarbonylamino, cyclobutylmethylcarbonylamino, cyclopentylmethylcarbonylamino, cyclohexylmethylcarbonylamino, methoxycarbonylamino, ethoxycarbonylamino, propoxycarbonylamino, 1-methylethoxycarbonylamino, 1,1-dimethyl-ethoxycarbonylamino, 2-methylpropoxycarbonylamino, 1-methylpropoxycarbonylamino, 2,2-dimethylpropoxycarbonylamino, 1,2-dimethylpropoxylcarbonylamino, or 1,1-dimethylpropoxy-carbonylamino.

(2) Within the groups (A), (B), A(a), and B(a) and more preferred groups contained therein, a more preferred group of compounds is that wherein:

### Y¹ is a bond; Y² is —NHC(O)NH—; and R⁴ is alkyl, preferably tert-butyl.

(3) Within the groups (A), (B), A(a), and B(a) and more preferred groups contained therein, a more preferred group of compounds is that wherein:

### Y¹ is a bond; Y² is —C(O)NH—; R^{2a} is a heteroaryl ring; and R⁴ is as defined in the Summary of the Invention, preferably alkyl, more preferably tert-butyl.

(4) Within the groups (A), (B), A(a), and B(a) and more preferred groups contained therein, a more preferred group of compounds is that wherein:

### Y¹ is a bond; and Y² is —OC(O)NH—.

(5) Within the groups (A), (B), A(a), and B(a) and more preferred groups contained therein, a more preferred group of compounds is that wherein:

### Y¹ is a bond; and Y² is —NHC(O)NH—.

It should be noted that reference to the preferred embodiments set forth above includes all combinations of particular and preferred groups unless stated otherwise.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1999.

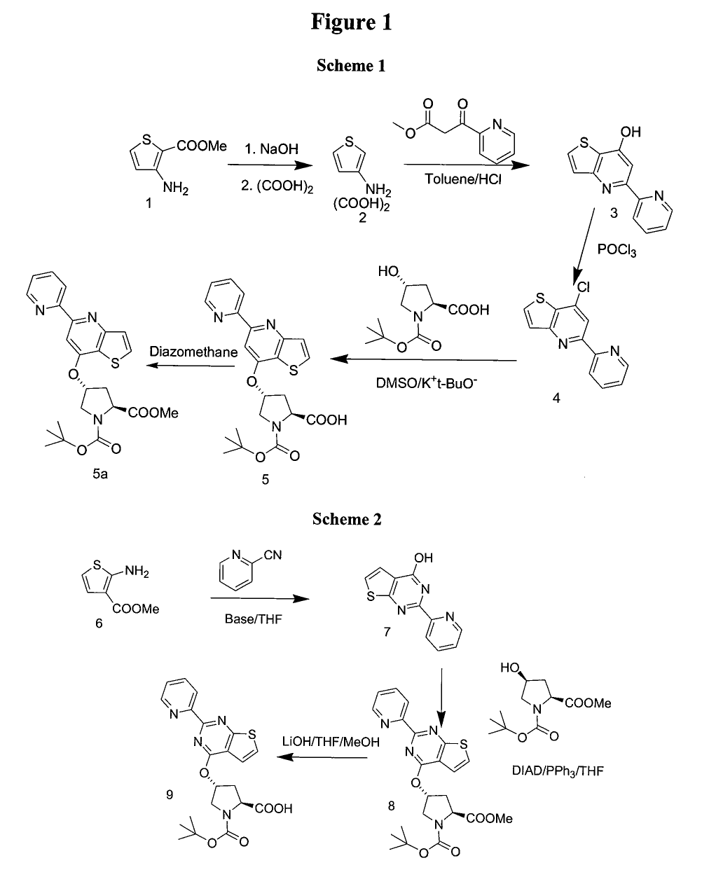

Schemes for intermediates in the preparation of compounds of Formula (I) are provided below, along with descriptions for the conversion of the intermediates to target compounds. In Scheme I (see FIG. 1), a pyridothiophene is prepared having a heteroaryl R^{2a} substituent and reacted to a hydroxyproline derivative to provide intermediate 5.

Conversion of 5 or 5a to target compounds can be accomplished using procedures as generally outlined in Example 14 of U.S. Ser. No. 11/478,337 (attorney docket no. 026503-006610US).

In a similar manner, Scheme 2 (see FIG. 1) illustrates the preparation of a heteroaryl substituted pyrimidinothiophene 7 and its conversion to key intermediates 8 and 9.

As above, conversion of 9 to target compounds can be accomplished using procedures as generally outlined in Example 14 of U.S. Ser. No. 11/478,337 (attorney docket no. 026503-006610US).

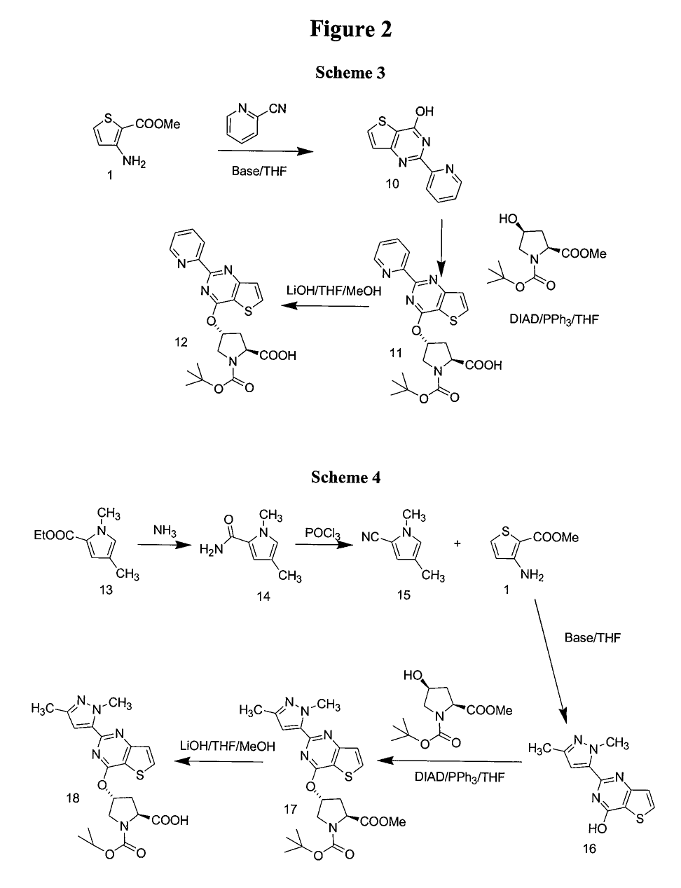

An isomeric pyrimidinothiophene intermediate is shown in Scheme 3 (see FIG. 2).

Scheme 4 (see FIG. 2) illustrates the preparation of a pyrazole-substituted pyrimidinothiophene 16 and its coupling with a proline derivative to form intermediates 17 and 18.

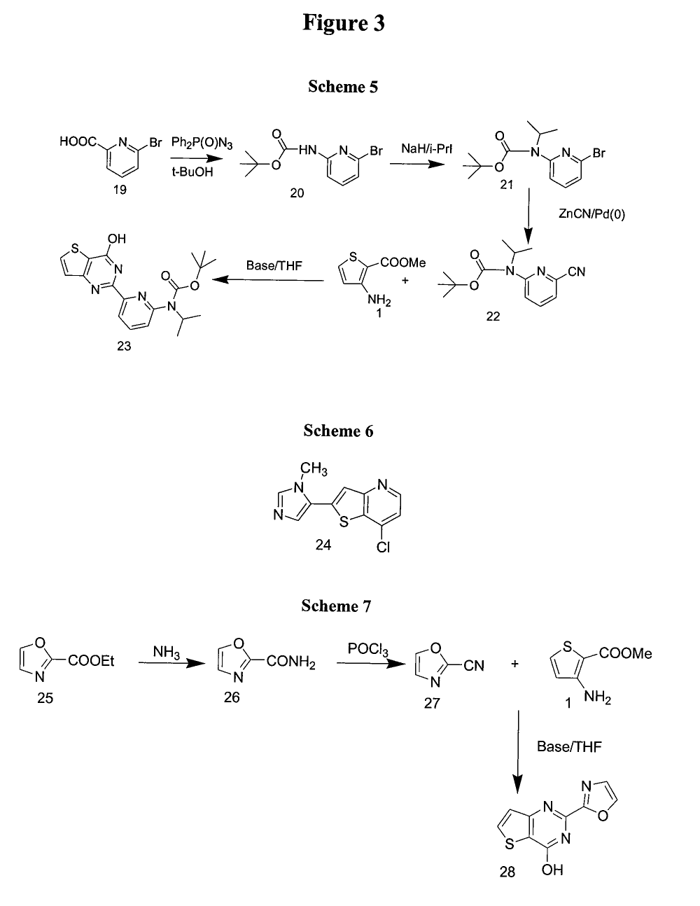

Scheme 5 (see FIG. 3) shows the preparation of pyridine-substituted pyrimidinothiophene 23, which can be reacted with a proline derivative to form intermediates analogous to 17 and 18 in Scheme 4 for incorporation into the final molecules.

Scheme 6 (see FIG. 3) shows intermediate 24 which is prepared as in WO 99/24440 and in U.S. Pat. No. 6,492,383 and used for incorporation into the final molecules as shown in Scheme 1.

Scheme 7 (see FIG. 3) shows the preparation of oxazole-substituted pyrimidinothiophene 28, which can be reacted with a proline derivative to form intermediates analogous to 17 and 18 in Scheme 4 for incorporation into the final molecules.

A compound of Formula (I) can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula (I) can be prepared by reacting the free acid form of the compound with a pharmaceutically acceptable inorganic or organic base. Inorganic and organic acids and bases suitable for the preparation of the pharmaceutically acceptable salts of compounds of Formula (I) are set forth in the definitions section of this Application. Alternatively, the salt forms of the compounds of Formula (I) can be prepared using salts of the starting materials or intermediates.

The free acid or free base forms of the compounds of Formula (I) can be prepared from the corresponding base addition salt or acid addition salt form. For example, a compound of Formula (I) in an acid addition salt form can be converted to the corresponding free base by treating with a suitable base (e.g., ammonium hydroxide solution, sodium hydroxide, and the like). A compound of Formula (I) in a base addition salt form can be converted to the corresponding free acid by treating with a suitable acid (e.g., hydrochloric acid, etc).

The N-oxides of compounds of Formula (I) can be prepared by methods known to those of ordinary skill in the art. For example, N-oxides can be prepared by treating an unoxidized form of the compound of Formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of Formula (I) can be prepared from the N-oxide of an appropriate starting material.

Compounds of Formula (I) in unoxidized form can be prepared from N-oxides of compounds of Formula (I) by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g., acetonitrile, ethanol, aqueous dioxane, or the like) at 0 to 80° C.

Prodrug derivatives of the compounds of Formula (I) can be prepared by methods known to those of ordinary skill in the art (e.g., for further details see Saulnier et al.(1994), Bioorganic and Medicinal Chemistry Letters, Vol. 4, p. 1985). For example, appropriate prodrugs can be prepared by reacting a non-derivatized compound of Formula (I) with a suitable carbamylating agent (e.g., 1,1-acyloxyalkylcarbonochloridate, para-nitrophenyl carbonate, or the like).

Protected derivatives of the compounds of Formula (I) can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds of the present invention may be conveniently prepared or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol.

Compounds of Formula (I) can be prepared as diastereomers that have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure isomer is then recovered by any practical means that would not result in racemization of its chiral centers. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Pharmacology and Utility

The compounds of the present invention are inhibitors of hepatitis C virus (HCV) replication and are therefore useful in treating hepatitis C infections. The inhibitory activities of the compounds of Formula (I) can be determined by methods known to those of ordinary skill in the art. A suitable in vitro assay for measuring the ability of compounds of this invention to inhibit HCV replication is set forth in Biological Example 1 infra.

Administration and Pharmaceutical Compositions

In general, compounds of Formula (I) will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula (I) may range from about 10 micrograms per kilogram body weight (μg/kg) per day to about 100 milligram per kilogram body weight (mg/kg) per day, typically from about 100 μg/kg/day to about 10 mg/kg/day. Therefore, a therapeutically effective amount for an 80 kg human patient may range from about 1 mg/day to about 8g/day, typically from about 1 mg/day to about 800 mg/day. In general, one of ordinary skill in the art, acting in reliance upon personal knowledge and the disclosure of this Application, will be able to ascertain a therapeutically effective amount of a compound of Formula (I) for treating a given disease.

The compounds of Formula (I) can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula (I) in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula (I) in the composition may vary widely depending upon the type of formulation, size of a unit dosage, kind of excipients and other factors known to those of skill in the art of pharmaceutical sciences. In general, a composition of a compound of Formula (I) for treating a given disease will comprise from 0.01% w to 90% w, preferably 5% w to 50% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula (I) are described below.

In some embodiments, the compounds of Formula (I) can be administered to a patient in need of treatment with a second antiviral agent. Examples of suitable antiviral agents are interferons, such as Intron A, Roferon A and pegylated interferons such as PEG-intron, Pegasys; Ribavirin, Viramidine, Levovirin; HCV polymerase inhibitors such as Valopicitabine, R 1626 (Roche), HCV-796 (Viropharma/Wyeth); and toll receptor agonists such as ANA-975 (Anadys). The compounds of Formula (I) can be administered in a combination with the above agents or separately. Additionally, the compounds of Formula (I) can be administered either prior to, or following, the administration of a second antiviral agent, according to a physician prescribed regimen.

### EXAMPLES

The present invention is further exemplified, but not limited by, the following examples that illustrate the preparation of compounds of Formula (I) according to the invention.

### Reference A

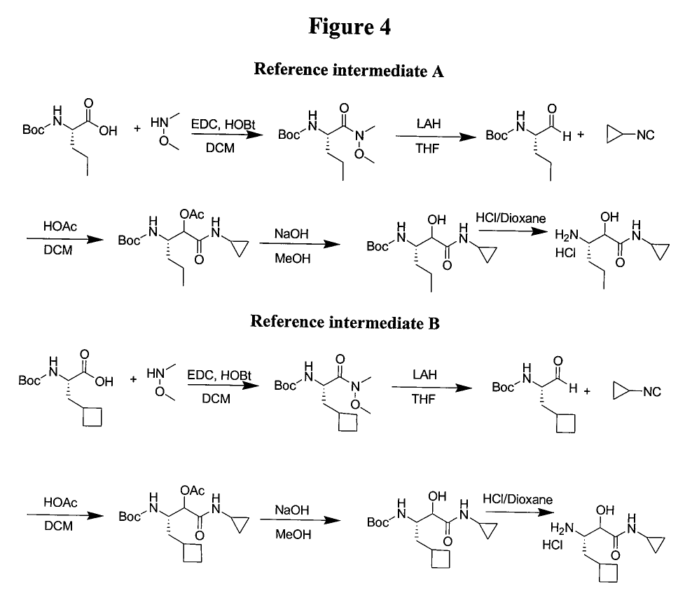

Synthesis of [1S-(cyclopropylcarbamoylhydroxymethyl)butyl]carbamic acid tert-butyl ester is provided in the scheme for Reference intermediate A (see FIG. 4).

### Step 1

To the mixture of Boc-NVa-OH (25 g, 0.115 mol), N,O-dimethylhydroxyamine hydrochloride (12.34 g, 0.127 mol), EDC (33.07 g, 0.173 mol), HOBt (22.9 g, 0.15 mol) in dichloromethane (300 mL), was slowly added NMM (34.9 g, 0.35 mol) under stirring in 30 min. The reaction was left at room temperature for 2 h, then diluted with 2000 mL EtOAc, washed with NaHCO₃, H₂O, and brine, and dried over MgSO₄. The solvent was removed on rotovap to give [1S-(methoxymethylcarbamoyl)butyl]carbamic acid tert-butyl ester (20g) as colorless oil.

### Step 2

To the solution of [1S-(methoxymethylcarbamoyl)butyl]carbamic acid tert-butyl ester (7.2 g, 27.7 mmol) in anhydrous THF (100 mL) under argon at −78° C., was slowly added LAH (1 M in THF, 27.7 mL). After 2 h, the reaction mixture was quenched by slowly adding 1N HCl (20 mL) and then allowed to warm up to room temperature. The reaction mixture was diluted with EtOAc (600 mL), washed with 1N HCl, H₂O, and brine and dried over MgSO₄. Removal of the solvents gave (1S-formylbutyl)carbamic acid tert-butyl ester (4.8 g) as an oil.

### Step 3

To a solution of cyclopropylisonitrile (1.91 g, 28.5 mmol), (1S-formylbutyl)carbamic acid tert-butyl ester (3.8 g, 19 mmol) in methylene chloride (100 mL) was added acetic acid (2.28 g, 38 mmol) at 0° C. After the addition was complete the reaction mixture was allowed to warm to 25° C. and stirred for 6 h. The reaction mixture was diluted with EtOAc (200 mL), then washed with satured solution of NaHCO₃ and brine (30 mL) and dried over MgSO₄. The solvent was removed and the crude product was crystallized from 50 mL of ethyl acetate and hexane(v/v=1/1) to give acetic acid 2-tert-butoxycarbonylamino-1-cyclopropylcarbamoylpentyl ester (4.8 g) as a white solid.

### Step 4

Into the solution of acetic acid 2-tert-butoxycarbonylamino-1-cyclopropylcarbamoyl-pentyl ester (4.8 g, 14.6 mmol) in methanol (50 mL) was added NaOH aqueous solution (1N, 22 mL) at room temperature. After 2 h, methanol was removed and the concentrate was extracted with ethyl acetate (300 mL). The ethyl acetate layer was washed with brine and dried over MgSO₄. After removal of the solvent, the crude product was crystallized from 100 mL of ethyl acetate and hexane(v/v=3/1) to give the title compound (3.5 g) as a white solid.

### Step 5

[1S-(cyclopropylcarbamoylhydroxymethyl)butyl]carbamic acid tert-butyl ester can then be converted into (3S)-3-amino-N-cyclopropyl-2-hydroxyhexanamide hydrochloride by treatment with 4.0 M HCl in dioxane in dichloromethane for 1 hr at rt followed by evaporation under reduced pressure.

### Reference B

Synthesis of (1S-cyclobutylmethyl-2-cyclopropylcarbamoyl-2-hydroxyethyl)-carbamic acid tert-butyl ester is provided in the scheme for Reference intermediate B (see FIG. 4).

### Step 1

To the mixture of Boc-L-cyclobutylalanine.DIPA (10.33 g, 30 mmol), N,O-dimethylhydroxyamine hydrochloride (3.22 g, 33 mmol), EDC (8.63 g, 45 mmol), HOBt (5.52 g, 36 mmol) in dichloromethane (200 mL), was slowly added NMM (9.11 g, 90 mmol) with stirring over 30 min. After 2 h, the reaction mixture was diluted with EtOAc (1000 mL), washed with NaHCO₃, H₂O, and brine and dried over MgSO₄. Removal of the solvent gave [2-cyclobutyl-1S-(methoxymethylcarbamoyl)ethyl]-carbamic acid tert-butyl ester (7.1 g) as a colorless oil.

### Step 2

To the solution of [2-cyclobutyl-1S-(methoxymethylcarbamoyl)ethyl]-carbamic acid tert-butyl ester (4.3 g, 15 mmol) in anhydrous THF (100 mL) under argon at −78° C., was slowly added LAH (1 M in THF, 15 mL, 15 mmol). After 2 h, the reaction mixture was quenched by slowly adding 1N HCl (15 mL) and the reaction mixture was warmed up to room temperature after the addition was complete. The reaction mixture was diluted with EtOAc (500 mL), washed with 1N HCl, H₂O, and brine and dried over MgSO₄. Removal of the solvents gave (2-cyclobutyl-1S-formylethyl)carbamic acid tert-butyl ester (2.95 g) as an oil.

### Step 3

To a solution of cyclopropylisonitrile (1.21 g, 18 mmol), (2-cyclobutyl-1S-formylethyl)carbamic acid tert-butyl ester (2.95 g, 13 mmol) in methylene chloride (20 mL), was added acetic acid (1.56 g, 26 mmol) at 0° C. After the addition was complete, the reaction mixture was allowed to warm to 25° C. and stirred for another 4 h. The reaction mixture was diluted with 200 mL EtOAc and washed with saturated solution of NaHCO₃ and brine and dried over MgSO₄. The solvent was removed and the crude product was crystallized from 50 mL of ethyl acetate and hexane (v/v=i/i) to give acetic acid 2S-tert-butoxycarbonylamino-3-cyclobutyl-1-cyclopropylcarbamoylpropyl ester (3.8 g) as a white solid.

### Step 4

To a solution of acetic acid 2S-tert-butoxycarbonylamino-3-cyclobutyl-1-cyclopropylcarbamoylpropyl ester (3.8 g, 10.7 mmol) in methanol (50 mL) was added NaOH aqueous solution (1N, 15 mL) at room temperature. After 2 h, methanol was removed and the concentrate was extracted with ethyl acetate. The ethyl acetate was washed with brine and dried over MgSO₄. The solvent was removed and the residue was crystallized from 100 mL of ethyl acetate and hexane(v/v=3/1) to give the title compound (2.9 g) as a white solid.

### Step 5

Tert-butyl (2S)-1-cyclobutyl-4-(cyclopropylamino)-3-hydroxy-4-oxobutan-2-ylcarbamate can then be converted into (3S)-3-amino-4-cyclobutyl-N-cyclopropyl-2-hydroxybutanamide hydrochloride by treatment with 4.0 M HCl in dioxane in dichloromethane for 1 hr at rt followed by evaporation under reduced pressure.

Intermediates 5, 9, 12, 18, 23, 24 and 28 (in Schemes 1-7, FIGS. 1-3) can be prepared as described in WO 2006/043145.

### Example 1

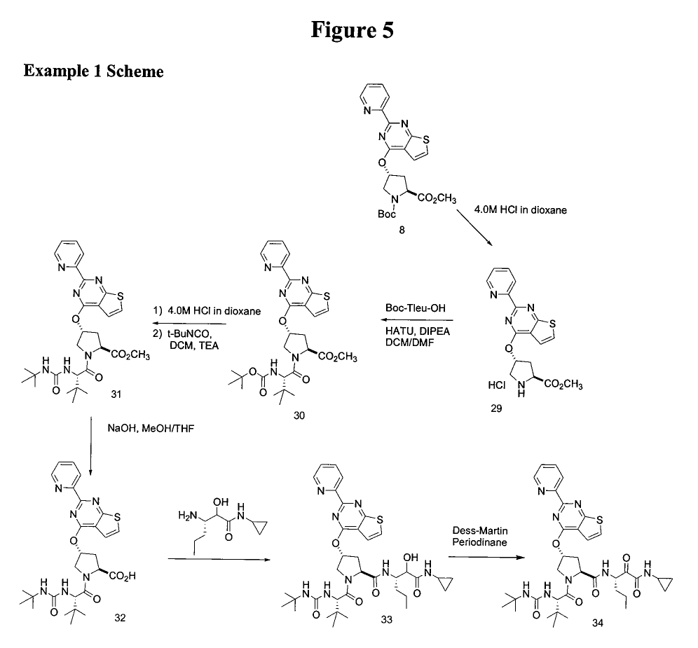

This example provides one synthesis route to (2S,4R)-1-((S)-2-(3-tert-butylureido)-3,3-dimethylbutanoyl)-N—((S)-1-(cyclopropylamino)-1,2-dioxohexan-3-yl)-4-(2-(pyridin-2-yl)thieno[2,3-d]pyrimidin-4-yloxy)pyrrolidine-2-carboxamide (34) (see Example 1 Scheme, FIG. 5).

### Step 1

(2S,4R)-1-tert-butyl 2-methyl 4-(2-(pyridin-2-yl)thieno[2,3-d]pyrimidin-4-yloxy)pyrrolidine-1,2-dicarboxylate (8) (1 mmol, prepared as shown in Scheme 2 and described in WO 2006/043145) can be converted into (29) by reaction with 4.0 M HCl in dioxane (6.0 mL) in dichloromethane (2 mL). After 1 h, evaporating the reaction mixture to dryness gives (2S,4R)-methyl 4-(2-(pyridin-2-yl)thieno[2,3-d]pyrimidin-4-yloxy)pyrrolidine-2-carboxylate hydrochloride (29).

### Step 2

(2S,4R)-methyl 4-(2-(pyridin-2-yl)thieno[2,3-d]pyrimidin-4-yloxy)pyrrolidine-2-carboxylate hydrochloride (29) can be converted into (30) by dissolving 0.165 mmol in dichloromethane/DMF (2.0 mL, 1:1) and adding Boc-L-tert-Leu-OH (0.165 mmol), HATU (0.182 mmol) and DIPEA (0.5 mmol) and stirring the mixture at rt. for 16 h. After diluting the reaction mixture with ethyl acetate, washing with 1N HCl, saturated NaHCO₃, and brine, separating the ethyl acetate layer, drying (MgSO₄), filtering and evaporating to dryness, (2S,4R)-methyl 1-((S)-2-(tert-butoxycarbonylamino)-3,3-dimethylbutanoyl)-4-(2-(pyridin-2-yl)thieno[2,3-d]pyrimidin-4-yloxy)pyrrolidine-2-carboxylate (30) can be isolated.

### Step 3

(2S,4R)-methyl 1-((S)-2-(tert-butoxycarbonylamino)-3,3-dimethylbutanoyl)-4-(2-(pyridin-2-yl)thieno[2,3-d]pyrimidin-4-yloxy)pyrrolidine-2-carboxylate (30) can be converted to (32) by first treating the crude material prepared in Step 2 with 4.0 M HCl in dioxane (3.0 mL) in dichloromethane (2 mL) to remove the t-Boc group and then, after evaporating the solvents under reduced pressure, reacting the resultant crude product with triethylamine (0.413 mmol) and tert-butylisocyanate (0.165 mmol) in dichloromethane (3.0 mL) at rt for 16 h. After aqueous/organic work up (diluting with dichloromethane and washing with 1N HCl, saturated NaHCO₃, and brine) and evaporating to dryness under reduced pressure, (2S,4R)-methyl 1-((S)-2-(3-tert-butylureido)-3,3-dimethylbutanoyl)-4-(2-(pyridin-2-yl)thieno[2,3-d]pyrimidin-4-yloxy)pyrrolidine-2-carboxylate (31) can be isolated.

### Step 4

(2S,4R)-methyl 1-((S)-2-(3-tert-butylureido)-3,3-dimethylbutanoyl)-4-(2-(pyridin-2-yl)thieno[2,3-d]pyrimidin-4-yloxy)pyrrolidine-2-carboxylate (31) can be converted into (32) by treatment with methanol (6.0 mL), THF (3.0 mL) and 1N NaOH (6 mL) for 1 h at rt. (2S,4R)-1-((S)-2-(3-tert-butylureido)-3,3-dimethylbutanoyl)-4-(2-(pyridin-2-yl)thieno[2,3-d]pyrimidin-4-yloxy)pyrrolidine-2-carboxylic acid (32) can then be isolated by aqueous/organic work up (concentrating the reaction mixture, acidifying with 1N HCl, extracting into ethyl acetate, washing with brine, drying over MgSO₄ and evaporating to dryness under reduced pressure).

### Step 5

(2S,4R)-1-((S)-2-(3-tert-butylureido)-3,3-dimethylbutanoyl)-4-(2-(pyridin-2-yl)thieno[2,3-d]pyrimidin-4-yloxy)pyrrolidine-2-carboxylic acid (32) (1 mmol) can be coupled with (3S)-3-amino-N-cyclopropyl-2-hydroxyhexanamide (1 mmol, as prepared in Reference A) in the presence of HATU (1.2 mmol) and diisopropylethyl amine (4 mmol) in dichloromethane and DMF to give (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(pyridin-2-yl)thieno[2,3-d]pyrimidin-4-yloxy)pyrrolidine-2-carboxamide (33) as a crude solid after aqueous/organic extractive work up.

### Step 6

Oxidizing (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(pyridin-2-yl)thieno[2,3-d]pyrimidin-4-yloxy)pyrrolidine-2-carboxamide (33) (1 mmol) with Dess-Martin periodinane (1.2 mmol) in dry dichloromethane can then provide (2S,4R)-1-((S)-2-(3-tert-butylureido)-3,3-dimethylbutanoyl)-N—((S)-1-(cyclopropylamino)-1,2-dioxohexan-3-yl)-4-(2-(pyridin-2-yl)thieno[2,3-d]pyrimidin-4-yloxy)pyrrolidine-2-carboxamide (34) after aqueous/organic work up and purification of the crude product on silica gel chromatography.

### Example 2

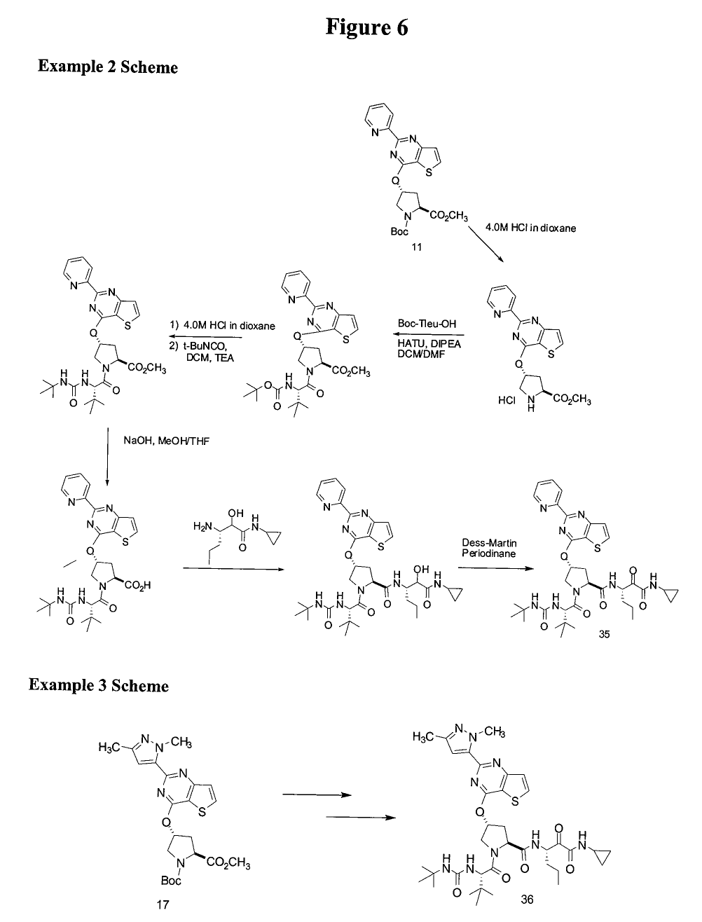

In a similar series of reactions to that shown in Example 1, (2S,4R)-1-tert-butyl 2-methyl 4-(2-(pyridin-2-yl)thieno[3,2-d]pyrimidin-4-yloxy)pyrrolidine-1,2-dicarboxylate (11) (as prepared in Scheme 3 and described in WO 2006/043145) can be converted into(2S,4R)-1-((S)-2-(3-tert-butylureido)-3,3-dimethylbutanoyl)-N—((S)-1-(cyclopropylamino)-1,2-dioxohexan-3-yl)-4-(2-(pyridin-2-yl)thieno[3,2-d]pyrimidin-4-yloxy)pyrrolidine-2-carboxamide (35) (see Example 2 Scheme, FIG. 6).

### Example 3

In a similar series of reactions to that shown in Example 1, (2S,4R)-1-tert-butyl 2-methyl 4-(2-(1,3-dimethyl-1H-pyrazol-5-yl)thieno[3,2-d]pyrimidin-4-yloxy)pyrrolidine-1,2-dicarboxylate (17) (as prepared in Scheme 4 and described in WO 2006/043145) can be converted into (2S,4R)-1-((S)-2-(3-tert-butylureido)-3,3-dimethylbutanoyl)-N—((S)-1-(cyclopropylamino)-1,2-dioxohexan-3-yl)-4-(2-(1,3-dimethyl-1H-pyrazol-5-yl)thieno[3,2-d]pyrimidin-4-yloxy)pyrrolidine-2-carboxamide (36) (see Example 3 Scheme, FIG. 6).

### Example 4

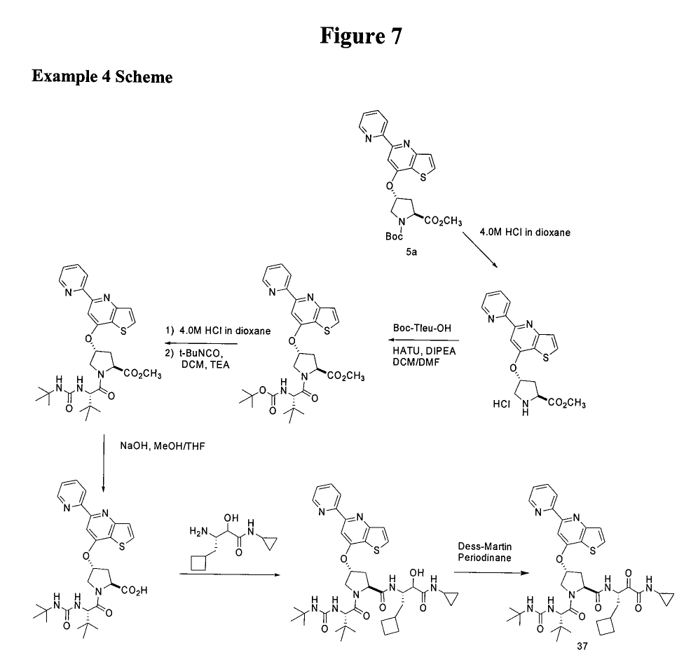

In a similar series of reactions to that shown in Example 1, (2S,4R)-1-tert-butyl 2-methyl 4-(5-(pyridin-2-yl)thieno[3,2-b]pyridin-7-yloxy)pyrrolidine-1,2-dicarboxylate (5a) (as prepared in Scheme 1 and described in WO 2006/043145) can be converted into (2S,4R)-1-((S)-2-(3-tert-butylureido)-3,3-dimethylbutanoyl)-N—((S)-1-cyclobutyl-4-(cyclopropylamino)-3,4-dioxobutan-2-yl)-4-(5-(pyridin-2-yl)thieno[3,2-b]pyridin-7-yloxy)pyrrolidine-2-carboxamide (37) by using (3S)-3-amino-4-cyclobutyl-N-cyclopropyl-2-hydroxybutanamide (as prepared in Reference B) in Step 5 (see Example 4 Scheme, FIG. 7).

### Example 5

This example illustrates an alternative approach to compounds of Formula I, having the heteroaryl component as provided in Example 4.

### Step 1: Preparation of 3-Aminothiophene oxalate salt

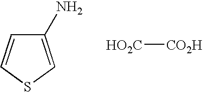

Methyl 3-aminothiophene-2-carboxylate (35 g, 220 mmol) was suspended in 1M NaOH (1. IL, 1.1 mol, 5 equiv.), the resulting mixture heated at reflux for 2 h then allowed to cool to room temperature overnight. After cooling to 0° C. conc. HCl (approx 90 mL) was added to acidify to pH 5. The resulting white precipitate was collected by filtration, taken-up in 1-propanol (350 mL) and treated with oxalic acid (39.6 g, 440 mmol, 2 equiv.). After stirring at 40° C. for 1 h the white precipitate was collected by filtration, and washed with Et₂O and then pentane (6.5 5g, 17%).

### Step 2: Preparation of Methyl 3-oxo-3-pyridin-2-ylpropanoate

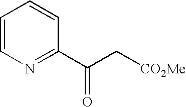

NaH (60% suspension on mineral oil; 30g, 750 mmol, 2.5 equiv.) was suspended in cyclohexane (900 mL) and the temperature raised to reflux. To this mixture was added 2-acetyl pyridine (36 g, 300 mmol) dropwise, followed, after 10 min, by dimethyl carbonate (100 mL, 1.2 mol, 4 equiv.). The reaction mixture was heated at reflux for 4 h, stood at room temperature overnight and then cooled to 0° C. The near solid mixture was quenched by addition of AcOH (75 mL) in H₂O (250 mL), then diluted with Et₂O (250 mL) and stirred until all solids had dissolved. The aqueous layer was separated and extracted with Et₂O (2×), the combined organic extracts dried (MgSO₄) and concentrated under reduced pressure. The crude orange oil was distilled to give recovered 2-acetyl pyridine (40° C./0.3 mbar; 6.5 g, 18%) and product (105° C./0.6 mbar; 27.5 g, 51%).

### Step 3: Preparation of 5-Pyridin-2-ylthieno[3,2-b]pyridin-7-ol

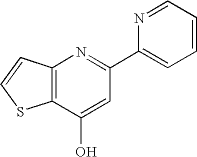

A mixture of 3-aminothiophene oxalate salt (6.5 g, 34.2 mmol) and methyl 3-oxo-3-pyridin-2-ylpropanoate (6.1 g, 1 equiv.) in toluene (100 mL) containing 4M HCl/dioxane (1 mL) was heated at reflux using Dean-Stark apparatus to remove H₂O for 20 h. After cooling to room temperature the grey precipitate was collected by filtration, washed with Et₂O and dried (9.4 g, quant.).

### Step 4: Preparation of 7-Chloro-5-pyridin-2-ylthieno[3,2-b]pyridine

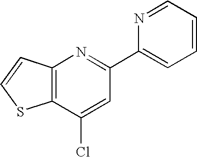

To 5-pyridin-2-ylthieno[3,2-b]pyridin-7-ol (4.82 g, 21.1 mmol) was added POCl₃ (80 mL). The temperature was gradually raised to 110° C. over 1 h, maintained at 100° C. for 1 h and the reaction mixture then concentrated under reduced pressure. The residue was partitioned between conc. aqueous K₂CO₃ (200 mL) and CHCl₃ (150 mL) and stirred at 11° C. for 2 h. The organic phase was separated, filtered through SiO₂ and concentrated under reduced pressure to yield a pale green oil (1.26 g, 25%).

### Step 5: Preparation of (2S,4R)-1-(tert-butoxycarbonyl)-4-(5-(pyridin-2-yl)thieno[3,2-b]pyridin-7-yloxy)pyrrolidine-2-carboxylic acid

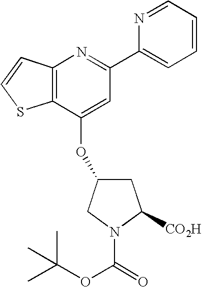

To N-Boc-4-hydroxy-L-proline (1.01 g, 4.37 mmol) in DMSO (30 mL) was added t-BuOK (1.11 g, 9.90 mmol, 2.27 equiv.). After 2 h a solution of 7-chloro-5-pyridin-2-ylthieno[3,2-b]pyridine (1.08 g, 1 equiv.) in DMSO (10 mL) was added in one portion. The reaction mixture was stirred at 30° C. for 90 h, then diluted with H₂O (180 mL) and washed with methyl tert-butyl ether (3×20 mL). The aqueous phase was acidified to pH 4.5 with 2M HCl then extracted with CH₂Cl₂ (3×20 mL). The combined organic extracts were washed with H₂O (2×20 mL), dried (Na₂SO₄) and concentrated under reduced pressure. The residue was purified by column chromatography (SiO₂, MeOH/CH₂Cl₂) to afford the product as a pale yellow solid (1.0 g, 57%).

### Step 6: Preparation of (2S,4R)-tert-butyl 2-((3S)-1-(cyclopropylamino)-2-hydroxy-1-oxohexan-3-ylcarbamoyl)-4-(5-(pyridin-2-yl)thieno[3,2-b]pyridin-7-yloxy)pyrrolidine-1-carboxylate

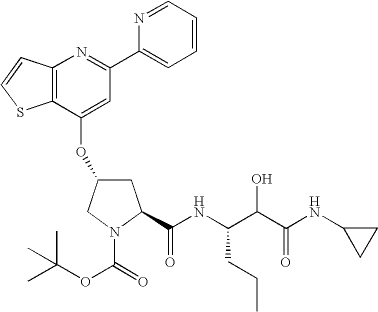

To a mixture of (2S,4R)-1-(tert-butoxycarbonyl)-4-(5-(pyridin-2-yl)thieno[3,2-b]pyridin-7-yloxy)pyrrolidine-2-carboxylic acid (150 mg, 340,mol), 3S-amino-N-cyclopropyl-2-hydroxyhexyramide hydrochloride (see Reference A above) (76 mg, 1 equiv.) and HATU (155 mg, 408 μmol, 1.2 equiv.) in CH₂Cl₂ (8 mL) and DMF (3 mL) was added iPr₂NEt (0.24 mL, 1.39 mmol, 4 equiv.). After 90 min at room temperature the reaction mixture was diluted with EtOAc (50 mL) and washed with 1M HCl (2×), sat. aqueous NaHCO₃ and brine. The organic phase was dried (MgSO₄) and concentrated under reduced pressure to give a yellow film (230 mg, quant).

### Step 7: Preparation of (2S,4R)-1-((S)-2-(3-tert-butylureido)-3,3-dimethylbutanoyl)-N-((3S)-1-(cyclopropylamino)-2-hydroxy-1-oxohexan-3-yl)-4-(5-(pyridin-2-yl)thieno[3,2-b]pyridin-7-yloxy)pyrrolidine-2-carboxamide.

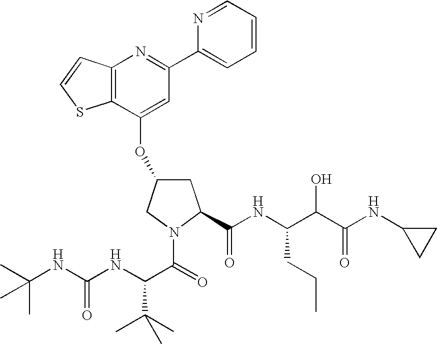

A mixture of (2S,4R)-tert-butyl 2-((3S)-1-(cyclopropylamino)-2-hydroxy-1-oxohexan-3-ylcarbamoyl)-4-(5-(pyridin-2-yl)thieno[3,2-b]pyridin-7-yloxy)pyrrolidine 1-carboxylate (max 340 μmol) in 4M HCl/dioxane (4 mL) and MeOH (2 mL) was stirred at room temperature for 90 min, then concentrated under reduced pressure and further concentrated from toluene (2×). To a mixture of this product, 2S-(3-tert-butylureido)-3,3-dimethylbutyric acid (78 mg, 1 equiv.) and HATU (155 mg, 408,mol, 1.2 equiv.) in CH₂Cl₂ (8 mL) and DMF (3 mL) was added iPr₂NEt (0.24 mL, 1.39 mmol, 4 equiv.). After 16 h at room temperature the reaction mixture was diluted with EtOAc (50 mL) and washed with 1M HCl (2×), sat. aqueous NaHCO₃ and brine. The organic phase was dried (MgSO₄) and concentrated under reduced pressure to give a buff solid foam (180 mg, 73%, three steps).

### Step 8: Preparation of (2S,4R)-1-((S)-2-(3-tert-butylureido)-3,3-dimethylbutanoyl)-N—((S)-1-(cyclopropylamino)-1,2-dioxohexan-3-yl)-4-(5-(pyridin-2-yl)thieno[3,2-b]pyridin-7-yloxy)pyrrolidine-2-carboxamide

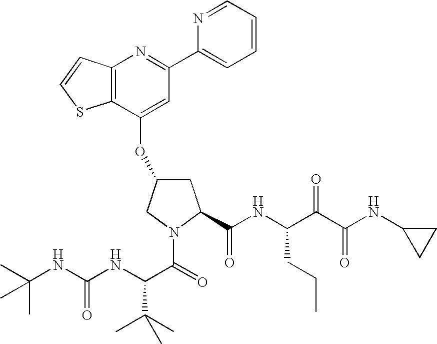

To a solution of (2S,4R)-1-((S)-2-(3-tert-butylureido)-3,3-dimethylbutanoyl)-N-((3S)-1-(cyclopropylamino)-2-hydroxy-1-oxohexan-3-yl)-4-(5-(pyridin-2-yl)thieno[3,2-b]pyridin-7-yloxy)pyrrolidine-2-carboxamide (165 mg, 229 mol) in CH₂Cl₂ (4 mL) was added Dess-Martin periodinane (195 mg, 460 μmol, 2 equiv.). After 2 h at room temperature the reaction mixture was treated with 0.26M Na₂S₂O₃ (5 mL) and sat. aqueous NaHCO₃ (5 mL), and the resulting mixture stirred vigorously for 30 min. The aqueous phase was separated and extracted with EtOAc (3×), the combined organic extracts washed with sat. aqueous NaHCO₃ (2×) and brine, then dried (MgSO₄) and concentrated under reduced pressure. Purification of the residue by column chromatography (SiO₂, EtOAc/hexanes) gave the product as a colourless powder (135 mg, 82%); ¹H NMR (400 MHz, DMSO-d₆) δ8.84 (1H, d), 8.68 (1H, d), 8.39 (1H, d), 8.28 (1H, d), 8.13 (1H, d), 8.01 (1H, t), 7.56-7.52 (2H, m), 7.48 (1H, d), 5.94-5.88 (2H, m), 5.77 (1H, br s), 5.07-5.00 (1H, m), 4.63 (1H, t), 4.23 (1H, d), 4.10-3.98 (2H, m), 2.78-2.73 (1H, m), 2.26-2.18 (1H, m), 1.75-1.68 (1H, m), 1.51-1.37 (3H, m), 1.07 (9H, s), 0.91 (9H, s), 0.88 (3H, t), 0.67-0.62 (2H, m) and 0.59-0.55 (2H, m); LCMS 100%: m/z 720 (MH⁺, 30%), 621 (60) and 508 (100).

### Example 6

This example illustrates the preparation of compounds having a 2-pyridin-2-ylthieno[2,3-c]pyrimidin-4-yl component.

### Step 1: Preparation of 2-Pyridin-2-ylthieno[2,3-c]pyrimidin-4-ol

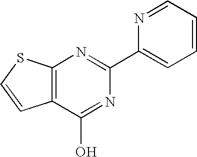

To a solution of methyl 2-aminothiophene-3-carboxylate (35 g, 220 mmol) and 2-cyanopyridine (23.2 g, 1 equiv.) in THF (800 mL) at 0° C. was added t-BuOK (37.5 g, 330 mmol, 1.5 equiv.) portionwise. After stirring at room temperature for 16 h the reaction mixture was concentrated under reduced pressure. The resulting dark solid residue was partitioned between CH₂Cl₂ (250 mL) and 25% w/v aqueous NH₄Cl (250 mL). The organic layer was separated, washed with H₂O and brine, then dried (Na₂SO₄) and concentrated under reduced pressure to give a solid that was triturated with methyl tert-butyl ether followed by acetone (6.95 g, 14%).

### Step 2: Preparation of 4-Chloro-2-pyridin-2-ylthieno[2,3-c]pyrimidine

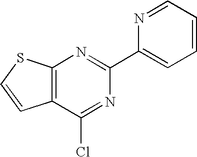

A mixture of 2-pyridin-2-ylthieno[2,3-c]pyrimidin-4-ol (6.9 g, 30 mmol) and POCl₃ (80 mL) was heated at 120-125° C. for 3.5 h, then concentrated under reduced pressure. The residue was partitioned between conc. aqueous K₂CO₃ (200 mL) and CHCl₃ (150 mL) and stirred at 10° C. for 1.5 h. The organic phase was separated, filtered through SiO₂ and concentrated under reduced pressure to yield a pale brown solid (5.73 g, 77%).

### Step 3: Preparation of (2S,4R)-1-(tert-butoxycarbonyl)-4-(2-(pyridin-2-yl)thieno[2,3-d]pyrimidin-4-yloxy)pyrrolidine-2-carboxylic acid

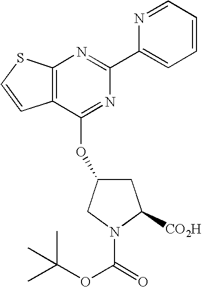

Prepared according to Example 5, Step 5, using 4-chloro-2-pyridin-2-ylthieno[2,3-c]pyrimidine (2.68 g, 10 mmol); following chromatography the product was obtained as an off-white solid (800 mg, 17%).

### Step 4: Preparation of (2S,4R)-tert-butyl 2-((3S)-1-(cyclopropylamino)-2-hydroxy-1-oxohexan-3-ylcarbamoyl)-4-(2-(pyridin-2-yl)thieno[2,3-d]pyrimidin-4-yloxy)pyrrolidine- 1-carboxylate

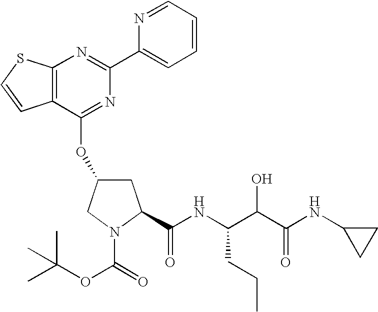

Prepared according to Example 5, Step 6, using (2S,4R)-1-(tert-butoxycarbonyl)-4-(2-(pyridin-2-yl)thieno[2,3-d]pyrimidin-4-yloxy)pyrrolidine-2-carboxylic acid (400 mg, 900 μmol); the product was obtained as an off-white solid (322 mg, 58%).

### Step 5: Preparation of (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(pyridin-2-yl)thieno[2,3-d]pyrimidin-4-yloxy)pyrrolidine-2-carboxamide

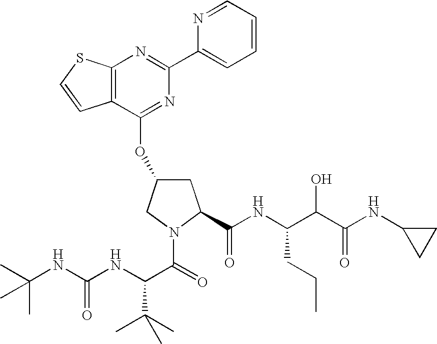

Prepared according to Example 5, Step 7, using (2S,4R)-tert-butyl 2-((3S)-1-(cyclopropylamino)-2-hydroxy-1-oxohexan-3-ylcarbamoyl)-4-(2-(pyridin-2-yl)thieno[2,3-d]pyrimidin-4-yloxy)pyrrolidine-1-carboxylate (420 mg, 688 mmol); the product was obtained as a buff solid (265 mg, 73%).

### Step 6: Preparation of (2S,4R)-1-((S)-2-(3-tert-butylureido)-3,3-dimethylbutanoyl)-N—((S)-1-(cyclopropylamino)-1,2-dioxohexan-3-yl)-4-(2-(pyridin-2-yl)thieno[2,3-d]pyrimidin-4-yloxy)pyrrolidine-2-carboxamide

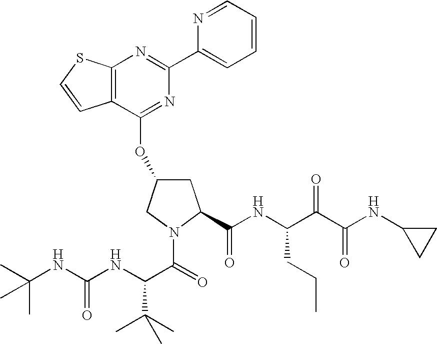

Prepared according to Example 5, Step 8, using (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(pyridin-2-yl)thieno[2,3-d]pyrimidin-4-yloxy)pyrrolidine-2-carboxamide (260 mg, 360 μmol); following chromatography the product was obtained as an off-white solid (136 mg, 52%); ¹H NMR (400 MHz, DMSO-d₆) δ8.78 (1H, d), 8.68 (1H, d), 8.48 (1H, d), 8.26 (1H, d), 7.99 (1H, t), 7.84 (1H, d), 7.53 (1H, t), 7.47 (1H, d), 5.96-5.88 (3H, m), 5.06-5.00 (1H, m), 4.63 (1H, t), 4.34 (1H, br d), 4.10 (1H, d), 4.06-4.02 (1H, m), 2.78-2.72 (1H, m), 2.64-2.56 (1H, m), 2.32-2.26 (1H, m), 1.75-1.68 (1H, m), 1.50-1.38 (2H, m), 1.10 (9H, s), 0.92 (9H, s), 0.88 (3H, t), 0.67-0.62 (2H, m) and 0.59-0.55 (2H, m); LCMS 100%: m/z 721 (MH⁺, 100%).

### Example 7

### Step 1: Preparation of 2-Pyridin-2-ylthieno[3,2-d]pyrimidin-4-ol

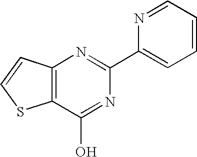

To a mixture of methyl 3-aminothiophene-2-carboxylate (31.4 g, 200 mmol) and 2-cyanopyridine (20.8 g, 1 equiv.) in THF (800 mL) at 0° C. was added tBuOK (33.7 g, 300 mmol, 1.5 equiv.). The reaction mixture was stirred at room temperature for 16 h then concentrated under reduced pressure. The solid residue was stirred into a mixture of CH₂Cl₂, sat. aqueous NH₄C₁ and H₂O (1:1:1; 600 mL) resulting in a suspension of a granular solid which was collected by filtration and dried (27.5 g, 60%).

### Step 2: Preparation of 4-Chloro-2-pyridin-2-ylthieno[3,2-d]pyrimidine

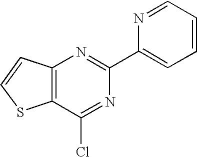

A mixture of 2-pyridin-2-ylthieno[3,2-d]pyrimidin-4-ol (23.4 g, 102 mmol) and POCl₃ (140 mL) was heated at reflux for 3 h then concentrated under reduced pressure. The residue was partitioned between CHCl₃ (350 mL) and conc. aqueous K₂CO₃ (500 mL). This mixture was stirred for 1.5 h, then filtered, and the phases separated. The organic phase was washed with brine, dried (MgSO₄) and concentrated under reduced pressure to afford the product as an oil (21.8 g, 87%).

### Step 3: Preparation of (2S,4R)-1-(tert-butoxycarbonyl)-4-(2-(pyridin-2-yl)thieno[3,2-d]pyrimidin-4-yloxy)pyrrolidine-2-carboxylic acid

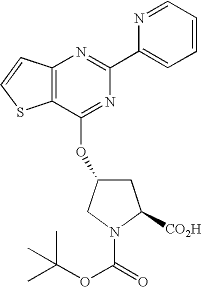

Prepared according to Example 5, Step 5, using 4-chloro-2-pyridin-2-ylthieno[3,2-d]pyrimidine (2.67 g, 10.8 mmol); following chromatography the product was obtained as an off-white solid (1.57 g, 33%).

### Step 4: Preparation of (2S,4R)-tert-butyl 2-((3S)-1-(cyclopropylamino)-2-hydroxy-1-oxohexan-3-ylcarbamoyl)-4-(2-(pyridin-2-yl)thieno[3,2-d]pyrimidin-4-yloxy)pyrrolidine 1-carboxylate

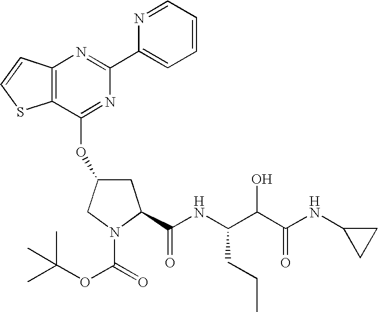

Prepared according to Example 5, Step 6 using (2S,4R)-1-(tert-butoxycarbonyl)-4-(2-(pyridin-2-yl)thieno[3,2-d]pyrimidin-4-yloxy)pyrrolidine-2-carboxylic acid (300 mg, 678 μmol); the product was obtained as a yellow solid (70 mg, 17%).

### Step 5: Preparation of (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(pyridin-2-yl)thieno[3,2-d]pyrimidin-4-yloxy)pyrrolidine-2-carboxamide

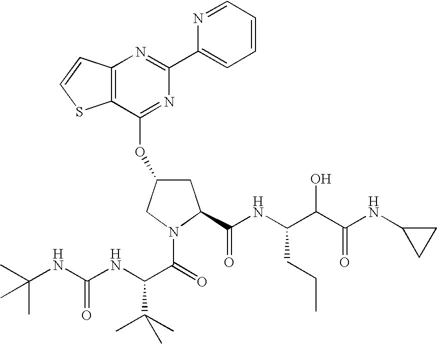

Prepared according to Example 5, Step 7, using (2S,4R)-tert-butyl 2-((3S)-1-(cyclopropylamino)-2-hydroxy-1-oxohexan-3-ylcarbamoyl)-4-(2-(pyridin-2-yl)thieno[3,2-d]pyrimidin-4-yloxy)pyrrolidine-1-carboxylate (70 mg, 115 μmol); the product was obtained as a buff solid (60 mg, 72%).

### Step 6: Preparation of (2S,4R)-1-((S)-2-(3-tert-butylureido)-3,3-dimethylbutanoyl)-N—((S)-1-(cyclopropylamino)-1,2-dioxohexan-3-yl)-4-(2-(pyridin-2-yl)thieno[3,2-d]pyrimidin-4-yloxy)pyrrolidine-2-carboxamide

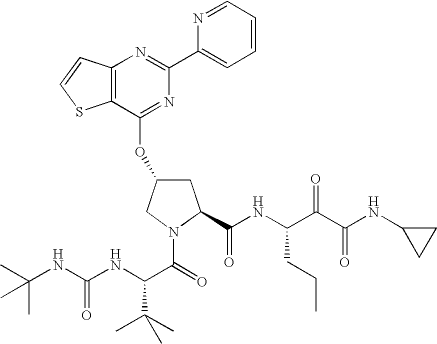

Prepared according to Example 5, Step 8 using (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(pyridin-2-yl)thieno[3,2-d]pyrimidin-4-yloxy)pyrrolidine-2-carboxamide (120 mg, 166 μmol); following chromatography the product was obtained as an off-white solid (60 mg, 50%); ¹H NMR (400 MHz, DMSO-d₆) δ8.78 (1H, d), 8.68 (1H, d), 8.47 (1H, d), 8.39 (1H, d), 8.31 (1H, d), 7.98 (1H, t), 7.67 (1H, d), 7.53 (1H, t), 6.03-5.97 (1H, m), 5.88 (1H, d), 5.84 (1H, s), 5.05-5.00 (1H, m), 4.65 (1H, t), 4.30 (1H, br d), 4.19-4.10 (3H, m), 2.78-2.71 (1H, m), 2.34-2.27 (1H, m), 1.74-1.68 (1H, m), 1.50-1.39 (3H, m), 1.02 (9H, s), 0.90 (9H, s), 0.87 (3H, t), 0.67-0.62 (2H, m) and 0.59-0.55 (2H, m); LCMS 97%: m/z 721 (MH⁺, 100%).

### Example 8

### Step 1: Preparation of 2-(1,3-dimethyl-1H-pyrazol-5-yl)thieno[3,2-d]pyrimidin-4-ol

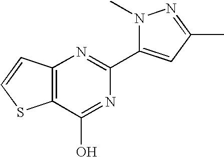

To a mixture of methyl 3-aminothiophene-2-carboxylate (9.1 g, 57.8 mmol) and 1,3-dimethylpyrazole-5-carbonitrile (7.0 g, 1 equiv.) in THF (300 mL) was added tBuOK (9.7 g, 86.7 mmol, 1.5 equiv.). After stirring at room for 16 h the reaction mixture was concentrated under reduced pressure, and the residue was poured into sat. aqueous NH₄Cl (300 mL). The resulting precipitate was collected by filtration, washed with H₂O, EtOH and Et₂O then dried (9.8 g, 70%).

### Step 2: Preparation of 4-chloro-2-(1,3-dimethyl-1H-pyrazol-5-yl)thieno[3,2-d]pyrimidine

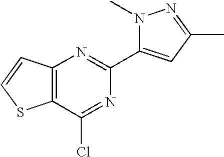

POCl₃ (25 mL) was added to 2-(1,3-dimethyl-1H-pyrazol-5-yl)thieno[3,2-d]pyrimidin-4-ol (5.6 g, 22.7 mmol) and the mixture was heated at reflux for 1.5 h. After cooling the reaction mixture was added to CHCl₃ (250 mL) and conc. aqueous K₂CO₃ (250 mL) at 10° C. After stirring for 1 h the organic phase was separated and washed with brine, dried (Na₂SO₄) and concentrated under reduced pressure to give a pale orange powder (4.78 g, 80%).

### Step 3: Preparation of (2S,4R)-1-(tert-butoxycarbonyl)-4-(2-(1,3-dimethyl-1H-pyrazol-5-yl)thieno[3,2-d]pyrimidin-4-yloxy)pyrrolidine-2-carboxylic acid

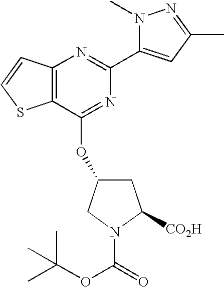

Prepared according to Example 5, Step 5 using 4-chloro-2-(1,3-dimethyl-1H-pyrazol-5-yl)thieno[3,2-d]pyrimidine (4.70 g, 17.8 mmol); following chromatography the product was obtained as an off-white solid (2.47 g, 31%).

### Step 4: Preparation of (2S,4R)-tert-butyl 2-((S)-1-(cyclopropylamino)-1,2-dioxohexan-3-ylcarbamoyl)-4-(2-(1,3-dimethyl-1H-pyrazol-5-yl)thieno[3,2-d]pyrimidin-4-yloxy)pyrrolidine-1-carboxylate

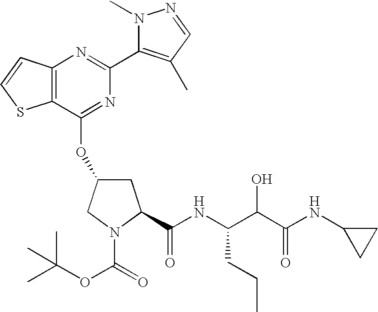

Prepared according to Example 5, Step 6 using (2S,4R)-1-(tert-butoxycarbonyl)-4-(2-(1,3-dimethyl-1H-pyrazol-5-yl)thieno[3,2-d]pyrimidin-4-yloxy)pyrrolidine-2-carboxylic acid (1.00 g, 2.18 mmol); the product was obtained as a yellow solid (1.40 g, quant).

### Step 5: Preparation of (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(1,3-dimethyl-1H-pyrazol-5-yl)thieno[3,2-d]pyrimidin-4-yloxy)pyrrolidine-2-carboxamide

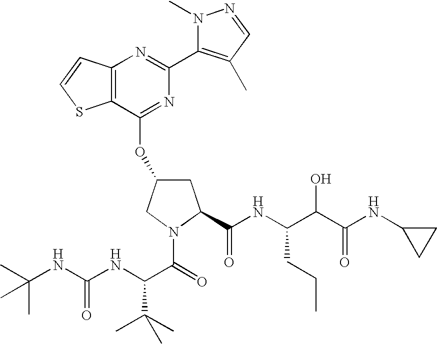

Prepared according to Example 5, Step 7 using (2S,4R)-tert-butyl 2-((S)-1-(cyclopropylamino)- 1,2-dioxohexan-3-ylcarbamoyl)-4-(2-(1,3-dimethyl-1H-pyrazol-5-yl)thieno[3,2-d]pyrimidin-4-yloxy)pyrrolidine-1-carboxylate (1.40 g, 2.10 mmol); the product was obtained as a buff solid (1.35 g, 90%).

### Step 6: Preparation of (2S,4R)-1-((S)-2-(3-tert-butylureido)-3,3-dimethylbutanoyl)-N—((S)-1-(cyclopropylamino)-1,2-dioxohexan-3-yl)-4-(2-(1,3-dimethyl-1H-pyrazol-5-yl)thieno[3,2-d]pyrimidin-4-yloxy)pyrrolidine-2-carboxamide

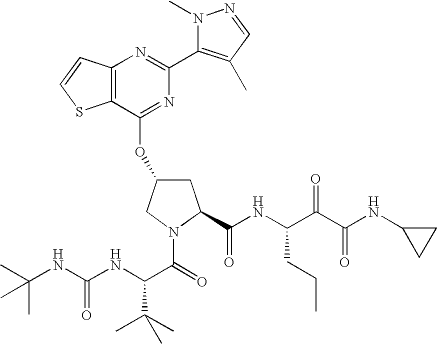

Prepared according to Example 5, Step 8 using (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(1,3-dimethyl-1H-pyrazol-5-yl)thieno[3,2-d]pyrimidin-4-yloxy)pyrrolidine-2-carboxamide (1.00 g, 1.35 mmol); following chromatography the product was obtained as an off-white solid (560 mg, 56%); ¹H NMR (400 MHz, DMSO-d₆) δ8.78 (1H, d), 8.37 (1H, d), 8.31 (1H, d), 7.59 (1H, d), 6.80 (1H, s), 5.92-5.86 (2H, m), 5.83 (1H, br s), 5.08-5.02 (1H, m), 4.63 (1H, t), 4.40 (1H, d), 4.23 (3H, s), 4.14 (1H, d), 4.05-3.99 (2H, m), 2.79-2.72 (1H, m), 2.30-2.23 (1H, m), 2.20 (3H, s), 1.75-1.69 (1H, m), 1.50-1.39 (2H, m), 1.00 (9H, s), 0.91 (9H, s), 0.87 (3H, t), 0.67-0.62 (2H, m) and 0.59-0.55 (2H, m); LCMS 100%: m/z 738 (MH⁺, 30%), 639 (45) and 526 (100).

### Example 9

### Step 1: Preparation of (S)-(9H-fluoren-9-yl)methyl 3-cyclobutyl-1-(methoxy(methyl)amino)-1-oxopropan-2-ylcarbamate

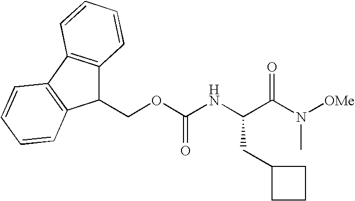

NMM (6.7 mL, 60.8 mmol, 3 equiv.) was added to a mixture of Fmoc-L-cyclobutylalanine (7.4 g, 20.3 mmol), N,O-dimethylhydroxylamine hydrochloride (2.17 g, 22.3 mmol, 1.1 equiv.), EDC (5.82 g, 30.5 mmol, 1.5 equiv.) and HOBt (3.56 g, 26.4 mmol, 1.3 equiv.) in CH₂Cl₂ (56 mL) and DMF (21 mL). After stirring at room temperature for 16 h the reaction mixture was diluted with EtOAc (500 mL) and washed with sat. aqueous NaHCO₃ (2×), H₂O and brine, dried (MgSO₄) and concentrated under reduced pressure to give a viscous pale yellow oil (8.85 g, quant.).

### Step 2: Preparation of (S)-(9H-fluoren-9-yl)methyl 1-cyclobutyl-3-oxopropan-2-ylcarbamate

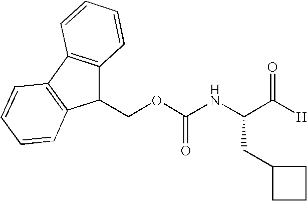

LiAlH₄ (1M in THF; 20 mL, 1 equiv.) was added dropwise to a solution of(S)-(9H-fluoren-9-yl)methyl 3-cyclobutyl-1-(methoxy(methyl)amino)-1-oxopropan-2-ylcarbamate (20.3 mmol) in THF (75 mL) at −78° C. The reaction mixture was stirred at −78° C. for 2 h, quenched by cautious addition of 1M HCl (15 mL) and allowed to warm to room temperature. EtOAc (500 mL) was added, the organic phase separated and washed with 1M HCl (2×), H₂O (2×) and brine (2×), dried (MgSO₄) and concentrated under reduced pressure to give a white solid (7.57 g, quant).

### Step 3: Preparation of (3S)-3-(((9H-fluoren-9-yl)methoxy)carbonylamino)-4-cyclobutyl-1-(cyclopropylamino)-1-oxobutan-2-yl acetate

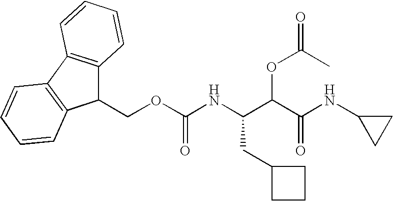

To a solution of (S)-(9H-fluoren-9-yl)methyl 1-cyclobutyl-3-oxopropan-2-ylcarbamate (20.3 mmol) in CH₂Cl₂ (100 mL) at 0° C. was added cyclopropylisonitrile (2.04 g, 30.4 mmol, 1.5 equiv.) followed by AcOH (2.3 mL, 40.6 mmol, 2 equiv.). The reaction mixture was stirred at 0° C. to room temperature over 16 h then diluted with EtOAc (400 mL) and washed with sat. aqueous NaHCO₃ (2×) and brine, dried (MgSO₄) and concentrated under reduced pressure to give a white solid (7.07 g, 73% over three steps).

### Step 4: Preparation of (9H-fluoren-9-yl)methyl (2S)-1-cyclobutyl-4-(cyclopropylamino)-3-hydroxy-4-oxobutan-2-ylcarbamate

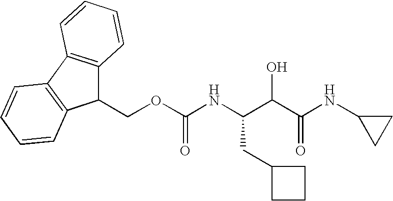

A solution of (3S)-3-(((9H-fluoren-9-yl)methoxy)carbonylamino)-4-cyclobutyl-1-(cyclopropylamino)-1-oxobutan-2-yl acetate (7.05 g, 14.8 mmol) in MeOH (75 mL) was treated with conc. H₂SO₄ (1 mL) and heated at reflux for 2 h. After cooling to room temperature H₂O (75 mL) was added, the resulting white precipitate was collected and dried (5.80 g, 90%).

### Step 5: Preparation of (3S)-3-amino-4-cyclobutyl-N-cyclopropyl-2-hydroxybutanamide

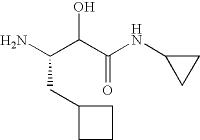

A solution of (9H-fluoren-9-yl)methyl (2S)-1-cyclobutyl-4-(cyclopropylamino)-3-hydroxy-4-oxobutan-2-ylcarbamate (5.30 g, 12.2 mmol) in 20% piperidine/DMF (50 mL) was stirred for 2 h then concentrated under high vacuum. The solid residue was dissolved in CH₂Cl₂ and loaded onto a short SiO₂ column. Elution with EtOAc removed non-polar impurities, elution with MeOH gave ninhydrin active fractions which were combined and concentrated to give the product as a white solid (2.45 g, 95%).

### Step 6: Preparation of N-(tert-Butoxycarbonyl)-L-tert-leucine

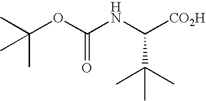

To a suspension of L-tert-leucine (5.0 g, 38 mmol) in dioxane (50 mL) and H₂O (35 mL) at 0° C. was added 1M NaOH (38 mL, 1 equiv.) followed by Boc₂O (9.3 g, 42 mmol, 1.1 equiv.). The reaction mixture was stirred at 0° C. to room temperature over 16 h then concentrated to approx half volume under reduced pressure. The aqueous residue was treated with EtOAc (75 mL) and 1 M HCl (50 mL), the aqueous phase separated and further extracted with EtOAc (50 mL). The combined organic extracts were dried (MgSO₄) and concentrated under reduced pressure to give the product as a colourless glassy solid (7.99 g, 92%).

### Step 7: Preparation of N-(tert-Butoxycarbonyl)-L-tert-leucine benzyl ester

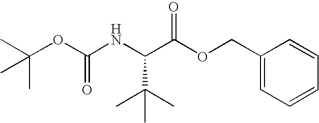

To a solution of N-(tert-butoxycarbonyl)-L-tert-leucine (7.9 g, 34 mmol) in MeCN (120 mL) at 0° C. was added benzyl bromide (4.5 mL, 37 mmol, 1.1 equiv.). DBU (6.1 mL, 40 mmol, 1.2 equiv.) was then added portionwise over 5 min. The reaction mixture was stirred at 0° C. to room temperature over 3 h then concentrated under reduced pressure. The residue was dissolved in EtOAc (80 mL) and washed with 1M HCl (2×40 mL), sat. aqueous NaHCO₃ (2×40 mL) and brine, then dried (MgSO₄) and concentrated under reduced pressure to afford the product (10.2 g, 93%).

### Step 8: Preparation of N-(Phenoxycarbonyl)-L-tert-leucine benzyl ester

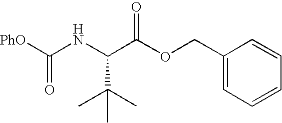

A solution of N-(tert-butoxycarbonyl)-L-tert-leucine benzyl ester (10.2 g, 31 mmol) in 2M HCl/dioxane (60 mL) was stirred at 40° C. for 4 h then concentrated under reduced pressure. The resulting hydrochloride salt was suspended in THF (62 mL), treated with pyridine (5.6 mL, 69.2 mmol, 2.25 equiv.), cooled to 0° C., then treated with phenyl chloroformate (4.1 mL, 32.6 mmol, 1.1 equiv.). After 1 h at room temperature the reaction mixture was diluted with EtOAc (200 mL) and washed with 1M HCl (75 mL), H₂O (75 mL), sat. aqueous NaHCO₃ (75 mL) and brine (75 mL), dried (MgSO₄) and concentrated under pressure to give the product as a pale yellow oil (9.65 g, 91% over two steps).

### Step 9: Preparation of (S)-benzyl 2-(3-(cyclopropylmethyl)ureido)-3,3-dimethylbutanoate

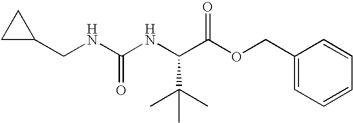

Cyclopropylmethylamine (0.36 mL, 4.15 mmol, 1.05 equiv.) was added to a solution of N-(Phenoxycarbonyl)-L-tert-leucine benzyl ester (1.37 g, 4.0 mmol) in DMSO (8 mL). After 1 h the reaction mixture was diluted with EtOAc (40 mL) and washed with H₂O (2×25 mL), 1M HCl (25 mL), H₂O (25 mL), 1M NaOH (25 mL) and brine (25 mL), then dried (MgSO₄) and concentrated under reduced pressure. The residue was purified by column chromatography (SiO₂, 30% EtOAc/hexanes) giving a pale yellow solid (1.12 g, 88%).

### Step 10: Preparation of (S)-2-(3-(cyclopropylmethyl)ureido)-3,3-dimethylbutanoic acid

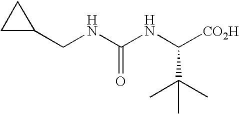

A mixture of (S)-benzyl 2-(3-(cyclopropylmethyl)ureido)-3,3-dimethylbutanoate (1.10 g, 3.45 mmol) and 10% Pd/C (110 mg) in EtOH (20 mL) was stirred under 1 atm. H₂ for 20 h, then filtered through celite and concentrated under reduced pressure to give the product as a white solid (760 mg, 96%).

### Step 11: Preparation of (2S,4R)-tert-butyl 2-((2S)-1-cyclobutyl-4-(cyclopropylamino)-3-hydroxy-4-oxobutan-2-ylcarbamoyl)-4-(2-(1,3-dimethyl-1H-pyrazol-5-yl)thieno[3,2-d]pyrimidin-4-yloxy)pyrrolidine-1-carboxylate

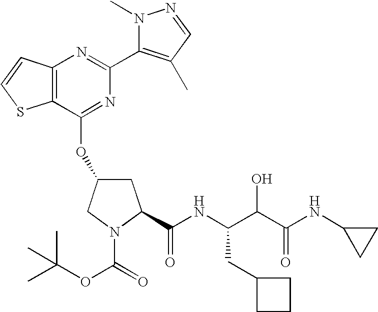

To a mixture of (2S,4R)-1-(tert-butoxycarbonyl)-4-(2-(1,3-dimethyl-1H-pyrazol-5-yl)thieno[3,2-d]pyrimidin-4-yloxy)pyrrolidine-2-carboxylic acid (200 mg, 435 μmol), (3S)-3-amino-4-cyclobutyl-N-cyclopropyl-2-hydroxybutanamide (92 mg, 1 equiv.) and HATU (199 mg, 522 μmol, 1.2 equiv.) in CH₂Cl₂ (8 mL) and DMF (4 mL) was added iPr₂NEt (0.23 mL, 1.31 mmol, 3 equiv.). After 1h at room temperature the reaction mixture was diluted with EtOAc (50 mL) and washed with 1M HCl (2×), sat. aqueous NaHCO₃ and brine. The organic phase was dried (MgSO₄) and concentrated under reduced pressure to give a yellow film (263 mg, 94%).

### Step 12: Preparation of (2S ,4R)-1-((S)-2-(3-tert-butylureido)-3,3-dimethylbutanoyl)-N-((3S)-1-(cyclopropylamino)-2-hydroxy-1-oxohexan-3-yl)-4-(2-(1,3-dimethyl-1H-pyrazol-5-yl)thieno[3,2-d]pyrimidin-4-yloxy)pyrrolidine-2-carboxamide

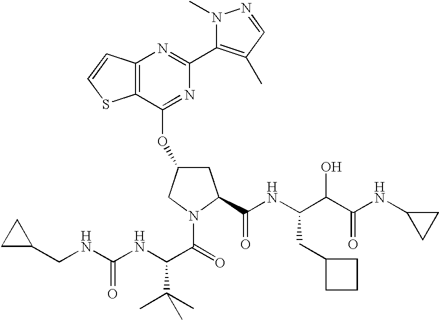

A mixture of (2S,4R)-tert-butyl 2-((2S)-1-cyclobutyl-4-(cyclopropylamino)-3-hydroxy-4-oxobutan-2-ylcarbamoyl)-4-(2-(1,3-dimethyl-1H-pyrazol-5-yl)thieno[3,2-d]pyrimidin-4-yloxy)pyrrolidine-1-carboxylate (260 mg, 397 μmol) in 4M HCl/dioxane (2 mL) and MeOH (0.2 mL) was stirred at room temperature for 2 h, then concentrated under reduced pressure and further concentrated from toluene (2×). To a mixture of this product, (S)-2-(3-(cyclopropylmethyl)ureido)-3,3-dimethylbutanoic acid (91 mg, 1 equiv.) and HATU (181 mg, 476 μmol, 1.2 equiv.) in CH₂Cl₂ (8 mL) and DMF (3 mL) was added iPr₂NEt (0.27 mL, 1.59 mmol, 4 equiv.). After 16 h at room temperature the reaction mixture was diluted with EtOAc (50 mL) and washed with 1M HCl (2×), sat. aqueous NaHCO₃ and brine. The organic phase was dried (MgSO₄) and concentrated under reduced pressure to give a buff solid foam (270 mg, 89% over two steps).

### Step 13: Preparation of (2S,4R)-1-((S)-2-(3-tert-butylureido)-3,3-dimethylbutanoyl)-N—((S)-1-(cyclopropylamino)-1,2-dioxohexan-3-yl)-4-(2-(1,3-dimethyl-1H-pyrazol-5-yl)thieno[3,2-d]pyrimidin-4-yloxy)pyrrolidine-2-carboxamide

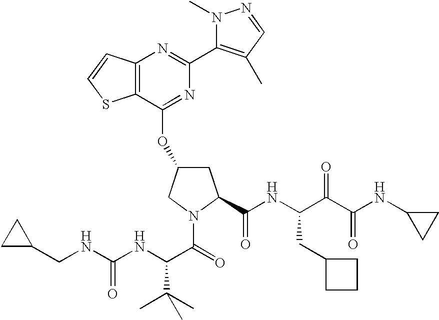

Prepared according to Example 5, Step 8 using (2S,4R)-1-((S)-2-(3-tert-butylureido)-3,3-dimethylbutanoyl)-N-((3S)-1-(cyclopropylamino)-2-hydroxy-1-oxohexan-3-yl)-4-(2-(1,3-dimethyl-1H-pyrazol-5-yl)thieno[3,2-d]pyrimidin-4-yloxy)pyrrolidine-2-carboxamide (250 mg, 327 μmol); following chromatography the product was obtained as a pale yellow powder (130 mg, 52%); ¹H NMR (400 MHz, DMSO-d₆) δ8.68 (1H, d), 8.38 (1H, d), 8.27 (1H, d), 7.61 (1H, d), 6.82 (1H, s), 6.06-6.00 (2H, m), 5.91 (1H, br s), 5.02-4.97 (1H, m), 4.63 (1H, t), 4.32 (1H, d), 4.23 (3H, s), 4.18 (1H, d), 4.10-4.01 (2H, m), 2.79-2.72 (1H, m), 2.63-2.57 (2H, m), 2.29-2.23 (1H, m), 2.20 (3H, s), 2.04-1.93 (2H, m), 1.85-1.72 (3H, m), 1.70-1.54 (3H, m), 0.92 (9H, s), 0.90-0.83 (1H, m), 0.68-0.62 (2H, m), 0.60-0.56 (2H, m), 0.32-0.27 (2H, m) and 0.03-0.00 (2H, m); LCMS 100%: m/z 762 (MH⁺, 20%), 552 (100) and 516 (30).

### BIOLOGICAL EXAMPLES

### Example 1

### HCV Replicon Assay

The HCV replicon assay is a cell-culture system that mimics in vivo HCV replication and provides a system to study HCV replication in vitro. It was created by transfecting cloned viral RNA derived from a consensus HCV genomic sequence into human Huh7 hepatoma cells that are semi-permissive for viral RNA production (Lohmann V., Korner F., Koch J.-0., Herian U., Theilmann L. and Bartenschlager R. (1999). Replication of subgenomic Hepatitis C virus RNAs in a hepatoma cell line. Science 285, 110-113 and Blight K. J., Kolykhalov A. A. and Rice C. M. (2000). Efficient initiation of HCV RNA Replication in cell culture. Science 290, 972-1974). These transfected cell lines contain a subgenomic HCV RNA genome that includes (1) the HCV 5′NTR fused to 12 amino acids of the capsid coding region, (2) the neomycin phosphotransferase gene (Neo) as a selectable marker, (3) the internal ribosome entry site (IRES) from encephalomyocarditis virus (EMCV) that directs translation of HCV non-structural proteins (variously NS2 or NS3 to NS5B), and (4) the 3′ NTR. Replicon-containing cells autonomously and persistently replicate HCV RNA that can be measured quantitatively by real-time qPCR. Therefore, the replicon system facilitates quantitative assessment of anti-viral activity by monitoring changes in HCV RNA replication in a cell-based assay.

HCV replicon-containing cells (Huh7/Clone A) were routinely maintained in Clone A growth medium (DMEM medium [Invitrogen], supplemented with 10% Fetal Bovine Serum, 1% Non Essential Amino Acids and 1 g/L G418). Test compounds were dissolved in dimethyl sulfoxide (DMSO) to make 200× stock solutions for all doses prior to treatment.

For the HCV replicon assay, Huh7/Clone A cells were trypsinized from culture flasks, seeded in 1 ml of Clone A growth medium without G418 at 4×10⁴ cells per well in 24-well plates and incubated at 37° C. in a humidified CO₂ (5%) incubator overnight. Following overnight incubation, compound solutions were added into wells in the same volume (5 μl of 200× compound stock per well) to give a final DMSO concentration of 0.5%. Three wells on each plate supplemented with 5 μl of DMSO served as untreated controls. For IC₅₀ determinations, compounds were tested at 7 serial dilutions in triplicates from the starting stock solutions. The plates were incubated at 37° C. for 48 hours. After incubation, cells were harvested, transferred to 96-well plates, and subjected to total RNA extraction using the RNA Isolation Kit (RNeasy 96, Qiagen) according to the protocol described by the manufacture's RNeasy 96 Handbook (Qiagen).

Total RNA eluted in 130 μl of RNase-free dH₂O was quantitated by the RiboGreen Assay according to the supplier's protocol (Molecular Probe). Briefly, 5 μl of RNA samples were aliquoted in duplicate to a 96-well black microplate and a 96-well TaqMan Optical plate. RNA samples in the black microplate were mixed with 95 μl of diluted RiboGreen reagent (1:250 dilution in TE buffer) and sample fluorescence was measured using a fluorescence microplate reader at standard fluorescein wavelengths (excitation ˜480 nm, emission ˜520 nm). Ribosomal RNA (Molecular Probe) was used as standard.

TaqMan quantitative PCR(RT-qPCR) was used to quantitate the amount of HCV replicon RNA in each sample. The RT-qPCR reactions were performed in 25 μl on an ABI PRISM 7900 HT Sequence Detection System (Applied Biosystems). The reaction mixture contained 5 μl of total RNA (10-100 ng), 1× TaqMan Buffer A (Applied Biosystems), 5.5 mM MgCl₂, 1.2 mM dNTP mix, 0.625 U of AmpliTaq Gold (Applied Biosystems), 5U of MMLV reverse transcriptase (Promega), 5 U of rRNasin (Promega), 300 nM each of the forward and reverse primers, and 100 nM TaqMan MGB probe. Primers and probe were designed to hybridize to a portion of the neomycin resistance gene (neo) in the replicon and the sequences are as follows: forward primer 5′-GGCTACCTGCCCATTCGA-3′; reverse primer 5′-CCGGCTTCCATCCGAGTAC-3′; MGB probe 5′-CCACCAAGCGAAACA-3′. The RT step was performed at 48° C. for 30 min, followed by 10 min at 95° C. The thermal cycling program consisted of 40 cycles of 15 s at 95° C. and 1 min at 60° C. TaqMan raw data (Ct values) were analyzed using the Sequence Detection System (SDS) software, mathematically converted to HCV RNA genome amount and normalized to total RNA in each sample. The sample without compound treatment served as a control and the HCV replicon RNA level from untreated cells was defined as 100%. Compound inhibitory activity was determined as the ratio of the normalized HCV RNA amount in treated samples relative to the untreated control. Compound IC₅₀s were calculated using a standard 4 parameter curve fit model.

Compounds of the invention prepared in Examples 5, 6, 7, 8 and 9 all inhibited HCV replication with IC₅₀s of less than 1 micromolar when evaluated using the assay above.

### Example 1

Representative pharmaceutical formulations containing a Compound of Formula (I)

ORAL FORMULATION

| | | |
|---|---|---|
| Compound of Formula (I) | 10-100 | mg |
| Citric Acid Monohydrate | 105 | mg |
| Sodium Hydroxide | 18 | mg |
| Flavoring | | |
| Water | q.s. to 100 | mL |

INTRAVENOUS FORMULATION

| Compound of Formula (I) | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

TABLET FORMULATION

| | |
|---|---|
| Compound of Formula (I) | 1% |
| Microcrystalline Cellulose | 73% |
| Stearic Acid | 25% |
| Colloidal Silica | 1% |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

What is claimed is:

1. A compound of Formula (I): 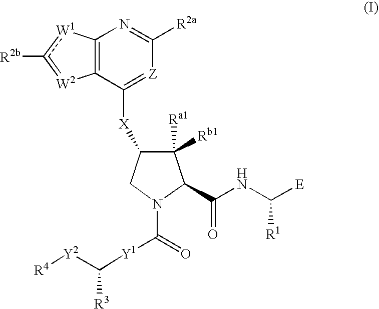
wherein
E is selected from the group consisting of-C(O)C(O)NR⁵R⁶, —C(O)CF₂C(O)NR⁵R⁶, —C(O)CF₂C(O)OR⁵, —C(O)C(O)R⁷, —C(O)CF₂R⁸, —C(O)R⁹, —C(O)C(O)OR¹⁰, —C(O)NR¹¹R¹², and —B(OR¹³)₂; wherein R⁵, R⁶, R⁷, R⁹, R¹⁰, R¹¹, R¹² and each R¹³ are independently selected from the group consisting of hydrogen, alkyl, alkenyl, alkynyl, cycloalkyl, cycloalkylalkyl, aryl, aralkyl, heteroaryl, heteroaralkyl, heterocyclyl, and heterocyclylalkyl; wherein R⁸ is selected from the group consisting of halo, alkyl, alkenyl, alkynyl, cycloalkyl, cycloalkylalkyl, aryl, aralkyl, heteroaryl, heteroaralkyl, heterocyclyl, and heterocyclylalkyl; wherein each aliphatic, alicyclic or aromatic moiety in R⁵, R⁶, R⁷, R⁸, R⁹, R¹⁰, R¹¹, R¹² or R¹³ is, optionally, independently substituted with one, two, or three R^{a}; wherein each R^{a} is independently selected from the group consisting of hydroxy, alkoxy, haloalkoxy, aryloxy, heteroaryloxy, alkylthio, arylthio, heteroarylthio, amino, monosubstituted amino, disubstituted amino, alkylsulfonyl, arylsulfonyl, carboxy, alkoxycarbonyl, alkenyloxycarbonyl, alkynyloxycarbonyl, alkenylaminocarbonyl, acylamino, aminocarbonyl, halo, and cyano; wherein each aromatic or alicyclic moiety in R^{a} is, optionally, independently substituted with one, two, or three R^{b}; wherein each R^{b} is independently selected from the group consisting of alkyl, alkoxy, halo, haloalkyl, haloalkoxy, cyano, carboxy, and carboxyalkyl; and wherein the two groups R⁵ and R⁶ or the two groups R¹¹ and R¹², in combination with the nitrogen to which the groups are attached, optionally form a five- to seven-membered heterocyclic or heteroaromatic ring;
W¹ and W² are each independently selected from the group consisting of CR^{2c}, O, S, N, and NR^{2d}; wherein the dashed line indicates the presence of one double bond, either between W¹ and CR^{2b} or between W² and CR^{2b};
X is selected from the group consisting of —O—, —NR^{2e}—, —S—, —SO—, and —SO₂—;
Z is selected from the group consisting of CH and N;
R¹ is independently selected from the group consisting of hydrogen, alkyl, haloalkyl, alkenyl, alkynyl, cycloalkyl, cycloalkylalkyl, aryl, aralkyl, heteroaryl, heteroaralkyl, heterocyclyl, and heterocyclylalkyl; wherein each aliphatic, alicyclic or aromatic moiety in R¹ is, optionally, independently substituted with one or two R^{c}; wherein each R^{c} is independently selected from the group consisting of hydroxy, alkoxy, aryloxy, heteroaryloxy, alkylthio, arylthio, heteroarylthio, amino, monosubstituted amino, disubstituted amino, alkylsulfonyl, arylsulfonyl, carboxy, alkoxycarbonyl, acylamino, aminocarbonyl, halo, and cyano; wherein each aromatic or alicyclic moiety in R^{c} is, optionally, independently substituted with one, two, or three R^{d}; and wherein each R^{d} is independently selected from the group consisting of alkyl, alkoxy, halo, haloalkyl, haloalkoxy, cyano, carboxy, and carboxyalkyl;
R^{2a}, R^{2b}, R^{2c}, R^{2d}, and R^{2e} are each independently selected from the group consisting of hydrogen, halo, cyano, alkyl, haloalkyl, alkenyl, alkynyl, —C(O)NR¹⁴R¹⁵, —OR¹⁴, —C(O)R¹⁴, —C(O)OR¹⁴, —OC(O)R¹⁴, —NR¹⁴C(O)R¹⁵, —NR¹⁴C(O)NR¹⁵R¹⁶, —NR¹⁴R¹⁵, —NR¹⁴OR¹⁵, —SO₂NR¹⁴R¹⁵, —NR¹⁴SO₂R¹⁵, aryl, heteroaryl, heterocyclyl, and cycloalkyl, provided that one of or both R^{2a} and R^{2b} are other than hydrogen, and further provided that both R^{2d} and R^{2e} are other than a member of the group consisting of halo, —OR¹⁴, —NR¹⁴OR¹⁵OC(O)R¹⁴, —NR¹⁴C(O)R¹⁵, —NR¹⁴C(O)NR¹⁵R¹⁶, —NR¹⁴R¹⁵, —NR¹⁴OR¹⁵; and —NR¹⁴SO₂R¹⁵; wherein each R¹⁴, R¹⁵, and R¹⁶ is independently selected from the group consisting of hydrogen, alkyl, cycloalkyl, cycloalkylalkyl, alkoxy, cycloalkoxy, alkenyl, alkynyl, aryl, and heterocyclyl; wherein each heterocyclic, alicyclic or aromatic moiety in R¹⁴, R¹⁵, or R¹⁶ is, optionally, independently substituted with one, two, three, four, or five R^{e}; wherein each R^{e} is independently selected from the group consisting of alkyl, alkenyl, alkynyl, halo, hydroxyl, alkoxy, cycloalkyl, cycloalkylalkyl, cycloalkyloxy, alkenyloxy, alkynyloxy, aryloxy, heteroaryloxy, alkylcarbonylamino, cycloalkylcarbonylamino, cycloalkylalkylcarbonylamino, alkyloxycarbonylamino, cycloalkyloxycarbonylamino, cycloalkylalkyloxycarbonylamino, amino, monosubstituted amino, disubstituted amino, alkylthio, arylthio, heteroarylthio, trifluoromethyl, sulfonyl, alkylsulfonyl, cycloalkylsulfonyl, arylsulfonyl, heteroarylsulfonyl, alkenylsulfonyl, alkynylsulfonyl, and heterocyclylsulfonyl; wherein each R^{e} is, optionally, independently substituted with one, two, or three R^{f}; and wherein each R^{f} is independently selected from the group consisting of alkyl, alkoxy, halo, haloalkyl, haloalkoxy, cyano, carboxy, and carboxyalkyl;
R³ is selected from the group consisting of hydrogen, alkyl, haloalkyl, alkenyl, alkynyl, cycloalkyl, cycloalkylalkyl, aryl, aralkyl, heteroaryl, heteroaralkyl, heterocyclyl, and heterocyclylalkyl; wherein each aliphatic, alicyclic or aromatic moiety in R³ is, optionally, independently substituted with one or two R^{g}; wherein each R^{g} is independently selected from the group consisting of hydroxy, alkoxy, haloalkoxy, aryloxy, heteroaryloxy, amino, monosubstituted amino, disubstituted amino, alkylthio, arylthio, heteroarylthio, alkylsulfonyl, arylsulfonyl, carboxy, alkoxycarbonyl, acylamino, aminocarbonyl, halo, and cyano; wherein each aromatic or alicyclic moeity in R^{g} is, optionally, substituted with one, two, or three R^{h}; and wherein each R^{h} is independently selected from the group consisting of alkyl, alkoxy, halo, haloalkyl, haloalkoxy, carboxy, or carboxyalkyl;
Y¹ is NR¹⁷ or a bond; wherein R¹⁷ is selected from the group consisting of hydrogen, alkyl, haloalkyl, cycloalkyl, and halocycloalkyl;
Y² is —C(O)NH—, —OC(O)NH—, —NR¹⁸—C(O)NH—, or —NR¹⁸C(O)O—; wherein each R¹⁸ is selected from the group consisting of hydrogen, alkyl, aryl, heteroaryl, or heterocyclyl; wherein each alkyl moiety in R¹⁷ is, optionally, independently substituted with one, two, or three Rⁱ groups; wherein each Rⁱ is independently selected from the group consisting of halo, hydroxyl, alkoxy, amino, monosubstituted amino, disubstituted amino, aryl, heteroaryl, and heterocyclyl; wherein each aromatic, heteroaromatic, or heterocyclic moeity in R¹⁷ or Rⁱ is, optionally, independently substituted with one, two, or three R^{j}; and wherein each R^{j} is chosen from the group consisting of halo and alkyl;
R⁴ is:
(i) alkyl, provided that Y¹ is a bond; Y² is —OC(O)NH—, —NR¹⁸—C(O)NH—, or —NR¹⁸C(O)O— and one, two, or three R^{j} are other than hydrogen;
(ii) selected from the group consisting of cycloalkyl, cycloalkylalkyl, aryl, aralkyl, heteroaryl, heteroaralkyl, heterocyclyl, and heterocyclylalkyl, provided that one or two members from the group consisting of R^{2a} and R^{2b} are independently selected heteroaryl when Y¹ is a bond; or
(iii) alkyl, provided that Y¹ is a bond; Y² is —C(O)NH— or —SO₂NH—, and one or two members from the group consisting of R^{2a} and R^{2b} are independently selected heteroaryl;
wherein each of the aromatic or alicyclic moieties in R⁴ is, optionally, independently substituted with one, two, or three R^{k}; wherein each R^{k} is independently selected from the group consisting of alkyl, alkoxy, halo, haloalkyl, haloalkoxy, cyano, carboxy, carboxyalkyl, hydroxyalkyl, alkoxyalkyl, aminoalkyl, alkylsulfonyl, alkylcarbonyl, aryl, aralkyl, arylsulfonyl, arylcarbonyl, aryloxycarbonyl, aminosulfonyl, aminocarbonyl, heteroaryl, heteroaralkyl, heteroarylsulfonyl, heteroarylcarbonyl, heteroaryloxycarbonyl, heterocyclyl, heterocyclylalkyl, heterocyclylsulfonyl, heterocyclylcarbonyl, heterocyclyloxycarbonyl, monosubstituted amino, and disubstituted amino; wherein each aromatic or alicyclic ring in R^{k} is, optionally, independently substituted with one, two, or three R^{m}; wherein each R^{m} is independently selected from the group consisting of alkyl, alkoxy, halo, haloalkyl, haloalkoxy, hydroxy, carboxy, alkoxycarbonyl, monosubstituted amino, disubstituted amino, and acylamino;
R^{a1} and R^{b1} are each independently selected from the group consisting of hydrogen, alkyl, cycloalkyl, and alkenyl; wherein each of the alkyl and cycloalkyl moieties in R^{a1} and R^{b1} are, optionally, independently substituted with one, two, or three Rⁿ moieties; wherein each Rⁿ moiety is independently selected from the group consisting of alkoxy, alkylthio, alkylsulfonyl, cycloalkyl, and halo;
wherein the compound has a molecular weight greater than 400 atomic mass units and less than 1100 atomic mass units; or
a pharmaceutically acceptable salt or solvate thereof.

2. The compound of claim 1, wherein W¹ is CR^{2c}; and wherein W² is S.

3. The compound of claim 2, wherein Z is N.

4. The compound of claim 3, wherein Y¹ is a bond; and wherein R^{a1} and R^{b1} are hydrogen.

5. The compound of claim 1, wherein W¹ is S; and wherein W² is CR^{2c}.

6. The compound of claim 5, wherein Z is N.

7. The compound of claim 6, wherein Y¹ is a bond; and wherein R^{a1} and R^{b1} are hydrogen.

8. The compound of claim 1, wherein R¹ is selected from the group consisting of alkyl and cycloalkylalkyl; wherein R³ is selected from the group consisting of 1-methylethyl, 1-methylpropyl, tert-butyl, cyclopropyl, phenyl, and cyclohexyl; wherein Y¹ is a bond; wherein R^{a1} and R^{b1} are hydrogen; and wherein X is —O—.

9. The compound of claim 8, wherein R¹ is selected from the group consisting of cyclopropylmethyl, cyclobutylmethyl, ethyl, and n-propyl; and wherein R³ is selected from the group consisting of tert-butyl and cyclohexyl.

10. The compound of claim 1, wherein E is —COCONR⁵R⁶; wherein each R⁵ and R⁶ is independently selected from the group consisting of hydrogen, alkyl, cycloalkyl, aralkyl, and heteroaralkyl; and wherein, optionally, R^{a} is halo.

11. The compound of claim 10, wherein R⁵ is hydrogen; and wherein R⁶ is selected from the group consisting of hydrogen and cyclopropyl.

12. The compound of claim 1, wherein Y¹ is a bond; wherein Y² is selected from the group consisting of —NR¹⁷C(O)NH— and —OC(O)NH—; and wherein R⁴ is alkyl.

13. The compound of claim 12, wherein Y² is —NHC(O)NH—; and wherein R⁴ is tert-butyl.

14. The compound of claim 1, wherein Y¹ is a bond; wherein R^{2a} is selected from the group consisting of aryl, heteroaryl, or heterocyclyl; wherein R^{2a} is, optionally, independently substituted with one, two, three, four, or five R^{e}; wherein each R^{e} is independently selected from the group consisting of halo, alkyl, cycloalkyl, cycloalkylalkyl, alkoxy, cycloalkoxy, alkylcarbonylamino, cycloalkylcarbonylamino, cycloalkylalkylcarbonylamino, alkyloxycarbonylamino, cycloalkyloxycarbonylamino, cycloalkylalkyloxycarbonylamino, amino, alkylamino, and dialkylamino, and —NHC(O)NR¹⁵R¹⁶; wherein R¹⁵ is independently selected from the group consisting of hydrogen and alkyl; wherein R¹⁶ is independently selected from the group consisting of hydrogen, alkyl, cycloalkyl, and cycloalkylalkyl; wherein each cycloalkyl or cycloalkylalkyl moiety in R¹⁶ is, optionally, independently substituted with one, two, or three alkyl; and wherein R^{2b} is selected from the group consisting of hydrogen, alkyl, aryl, and heteroaryl.

15. The compound of claim 14, wherein R^{2a} is selected from the group consisting of: 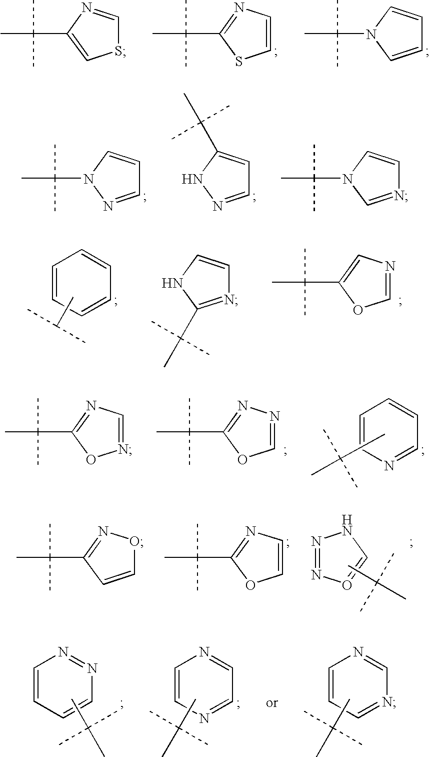
wherein R^{2a} is, optionally, independently substituted with one, two, or three R^{e}; wherein each R^{e} is independently selected from the group consisting of halo, alkyl, cycloalkyl, cycloalkylalkyl, alkoxy, cycloalkoxy, alkyloxycarbonylamino, amino, alkylamino, and dialkylamino, and —NHC(O)NR¹⁵R¹⁶; wherein R¹⁵ is independently selected from the group consisting of hydrogen and alkyl; wherein R¹⁶ is independently selected from the group consisting of hydrogen, alkyl, cycloalkyl, and cycloalkylalkyl; wherein each cycloalkyl or cycloalkylalkyl moiety in R¹⁶ is, optionally, independently substituted with one, two, or three alkyl; and wherein R^{2b} is selected from the group consisting of hydrogen, alkyl, aryl, and heteroaryl.

16. The compound of claim 15, wherein R^{2a} is selected from the group consisting of: 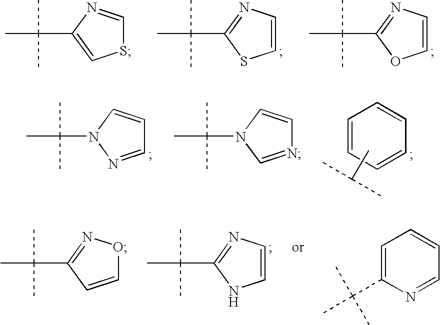
wherein R^{2a} is, optionally, independently substituted with one, two, or three R^{e}; wherein each R^{e} is independently selected from the group consisting of halo, alkyl, cycloalkyl, cycloalkylalkyl, alkoxy, cycloalkoxy, alkyloxycarbonylamino, amino, alkylamino, and dialkylamino, and —NHC(O)NR¹⁵R₁₆; wherein R¹⁵ is independently selected from the group consisting of hydrogen and alkyl; wherein R¹⁶ is independently selected from the group consisting of hydrogen, alkyl, cycloalkyl, and cycloalkylalkyl; wherein each cycloalkyl or cycloalkylalkyl moiety in R¹⁶ is, optionally, independently substituted with one, two, or three alkyl; and wherein R^{2b} is selected from the group consisting of hydrogen, alkyl, aryl, and heteroaryl.

17. The compound of claim 16, wherein R^{2a} is selected from the group consisting of: 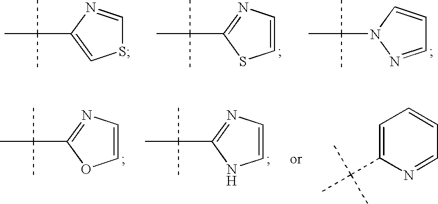
wherein R^{2a} is, optionally, independently substituted with one, two, or three R^{e}; wherein each R^{e} is independently selected from the group consisting of halo, alkyl, cycloalkyl, cycloalkylalkyl, alkoxy, cycloalkoxy, alkyloxycarbonylamino, amino, alkylamino, and dialkylamino, and —NHC(O)NR¹⁵R¹⁶; wherein R¹⁵ is independently selected from the group consisting of hydrogen and alkyl; wherein R¹⁶ is independently selected from the group consisting of hydrogen, alkyl, cycloalkyl, and cycloalkylalkyl; wherein each cycloalkyl or cycloalkylalkyl moiety in R¹⁶ is, optionally, independently substituted with one, two, or three alkyl; and wherein R^{2b} is selected from the group consisting of hydrogen, alkyl, aryl, and heteroaryl.

18. The compound of claim 14 wherein each optional R^{e} is independently selected from the group consisting of methyl, ethyl, n-propyl, iso-propyl, n-butyl, 1-methylpropyl, 2-methylpropyl, tert-butyl, 2,2-dimethylpropyl, 1,2-dimethylpropyl, 1,2,2-trimethylpropyl, cyclopropyl, cyclobutyl, cyclopentyl, cyclohexyl, cyclopropylmethyl, cyclobutylmethyl, cyclopentylmethyl, and cyclohexylmethyl.

19. The compound of claim 14 wherein each optional R^{e} is independently selected from the group consisting of amino, methylamino, ethylamino, propylamino, 1-methylethylamino, 1,1-dimethylethylamino, 2-methylpropylamino, 1-methylpropylamino, 2,2-dimethylpropylamino, 1,2-dimethylpropylamino, 1,1-dimethylpropylamino, cyclopropylamino, cyclobutylamino, cyclopentylamino, cyclohexylamino, cyclopropylmethylamino, cyclobutylmethylamino, cyclopentylmethylamino, cyclohexylmethylamino, methylcarbonylamino, ethylcarbonylamino, propylcarbonylamino, 1-methylethylcarbonylamino, 1,1-dimethylethylcarbonylamino, 2-methylpropylcarbonylamino, 1-methylpropylcarbonylamino, 2,2-dimethylpropylcarbonylamino, 1,2-dimethylpropylcarbonylamino, 1,1-dimethylpropylcarbonylamino, cyclopropylcarbonylamino, cyclobutylcarbonylamino, cyclopentylcarbonylamino, cyclohexylcarbonylamino, cyclopropylmethylcarbonylamino, cyclobutylmethylcarbonylamino, cyclopentylmethylcarbonylamino, cyclohexylmethylcarbonylamino, methoxycarbonylamino, ethoxycarbonylamino, propoxycarbonylamino, 1-methylethoxycarbonylamino, 1,1-dimethyl-ethoxycarbonylamino, 2-methylpropoxycarbonylamino, 1-methylpropoxycarbonylamino, 2,2-dimethylpropoxycarbonylamino, 1,2-dimethylpropoxylcarbonylamino, or 1,1-dimethylpropoxy-carbonylamino.

20. The compound of claim 14 wherein R^{2a} is selected from the group consisting of 2-pyridyl, 6-iso-propylamino-pyridin-2-yl, oxazol-2-yl, and 1,3-dimethyl-1H-pyrazol-4-yl; and wherein R^{2b} is selected from the group consisting of hydrogen and 3-methyl-3H-imidazol-4-yl.

21. A compound having a structure selected from the group consisting of: 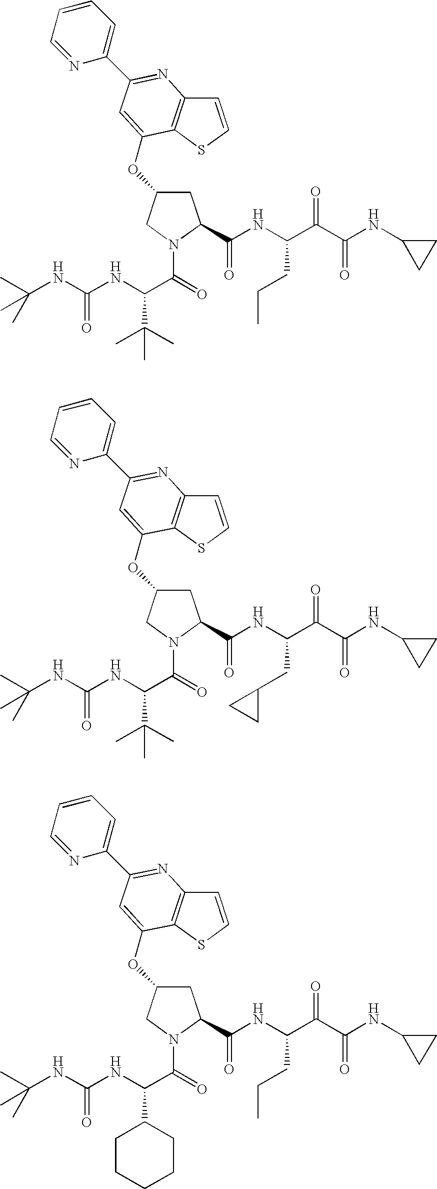 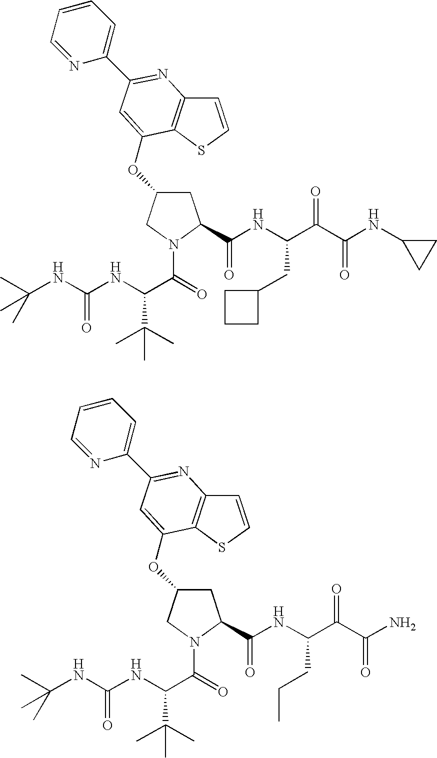
or a pharmaceutically acceptable salt thereof.

22. A compound selected from the group consisting of: 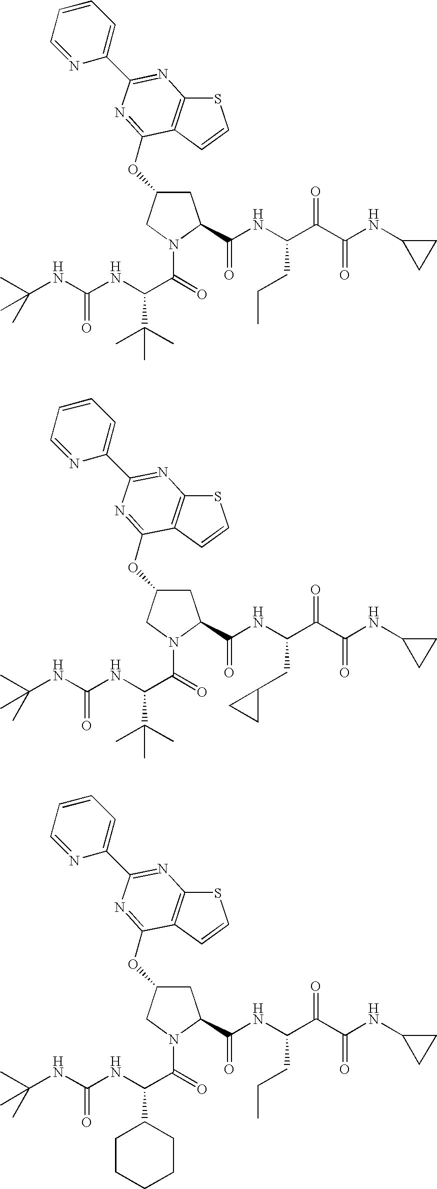 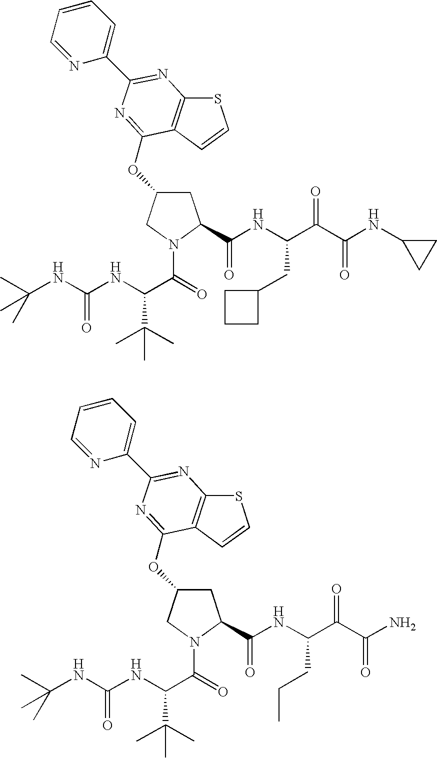
or a pharmaceutically acceptable salt thereof.

23. A compound selected from the group consisting of: 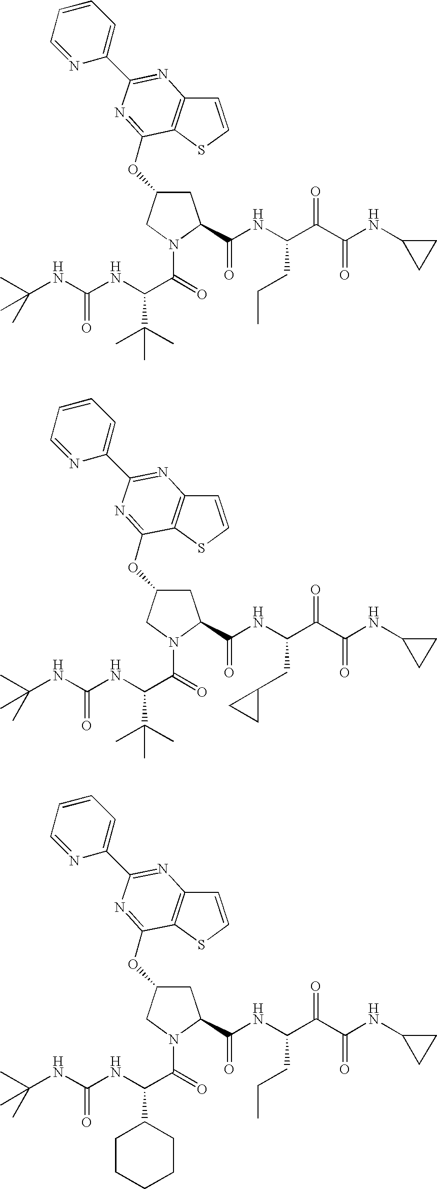 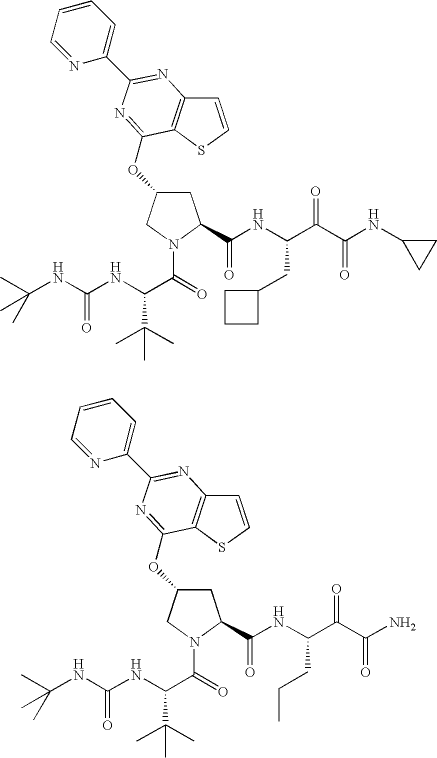
or a pharmaceutically acceptable salt thereof.

24. A compound selected from the group consisting of: 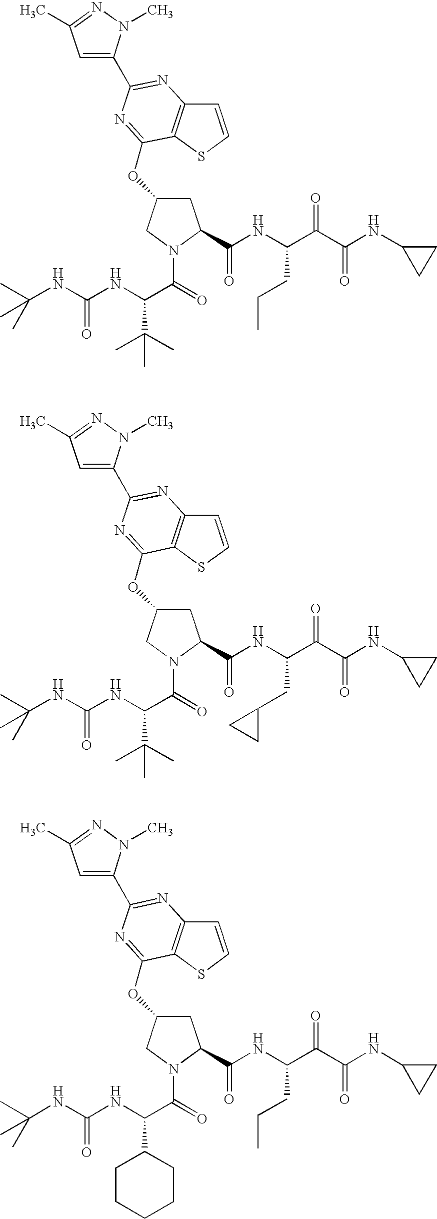 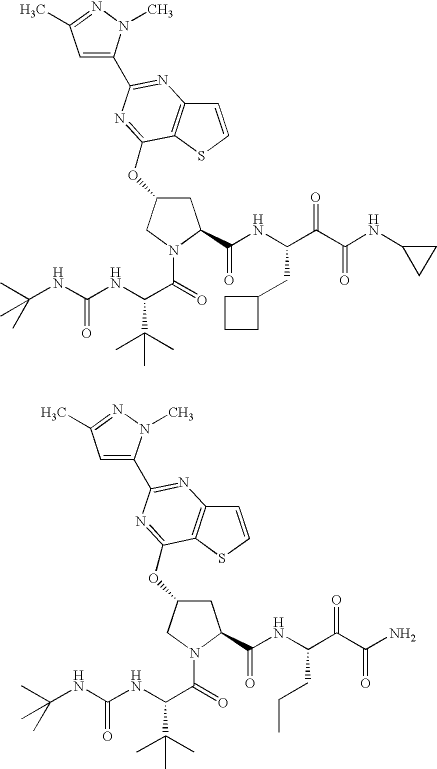
or a pharmaceutically acceptable salt thereof.

25. A compound selected from the group consisting of: 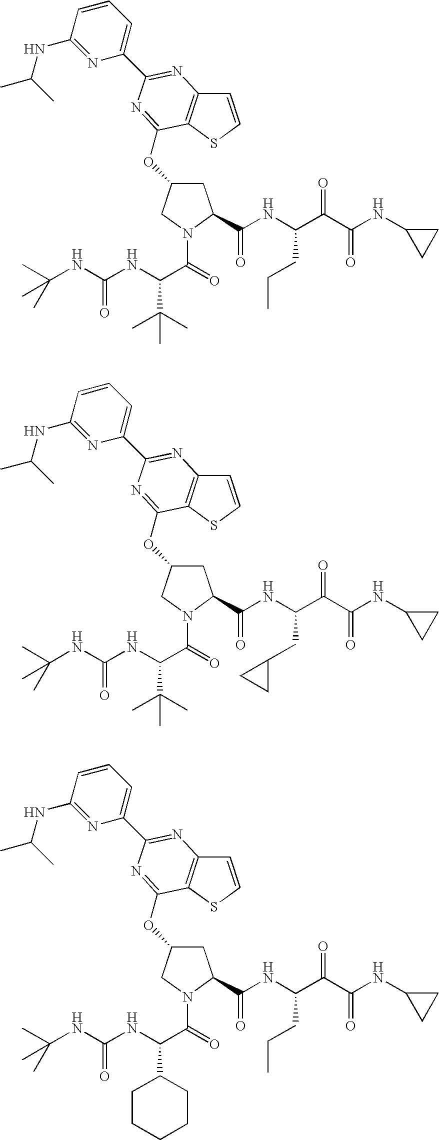 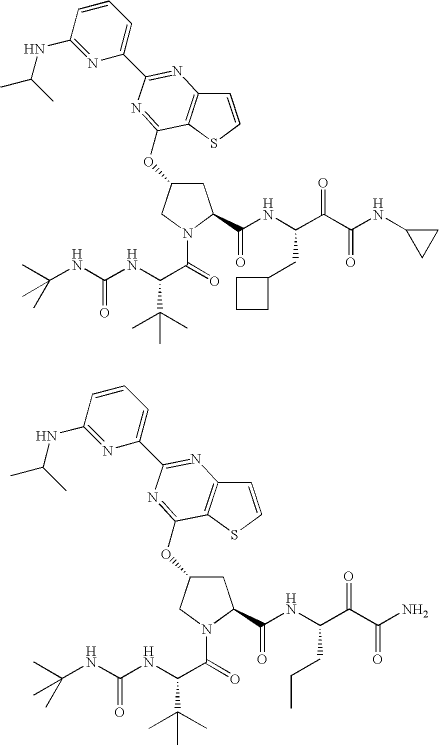
or a pharmaceutically acceptable salt thereof.

26. A compound selected from the group consisting of: 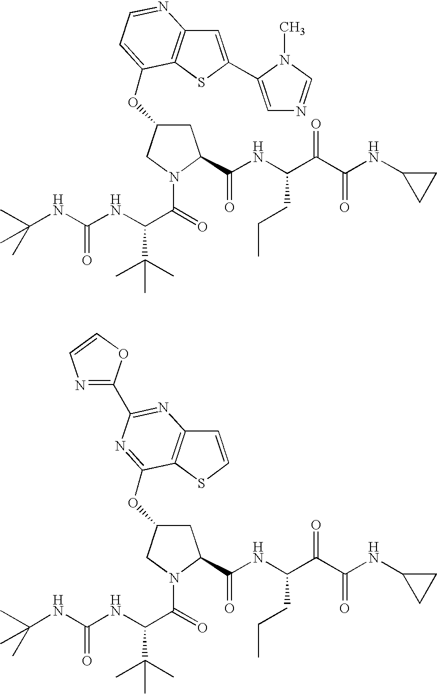
or a pharmaceutically acceptable salt thereof.

27. A compound of claim 1, having the formula:

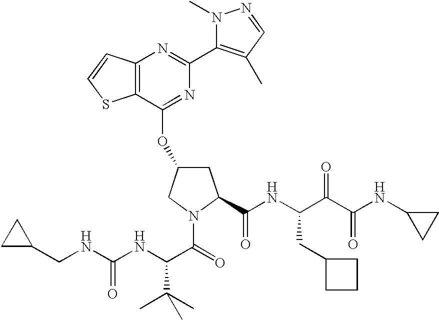

28. A pharmaceutical composition comprising a compound of claim 1 and one or more pharmaceutically acceptable excipients.

29. A method of treating hepatitis C infections in a patient comprising administering to the patient a pharmaceutical composition comprising the compound of claim 1 and one or more pharmaceutically acceptable excipients.

30. The pharmaceutical composition of claim 28, further comprising a second antiviral agent.

31. The pharmaceutical composition of claim 30, wherein said second antiviral agent is Ribavirin or a polymerase inhibitor.

32. A method of treating hepatitis C infections in a patient, comprising administering to said patient the compound of claim 1 and a second antiviral agent selected from the group consisting of interferon, pegylated or unpegylated congeners of interferon, Ribavirin, cyclosporine, a cyclosporine analog, a HCV polymerase inhibitor and a toll receptor agonist.
